(19) United States
(12) Patent Application Publication
(10) Pub. No.: US 2023/0312486 A1
(43) Pub. Date: Oct. 5, 2023

(54) POLYMORPHIC FORMS OF A ROR INHIBITING COMPOUND AND PROCESSES FOR ITS PREPARATION

(71) Applicant: Glenmark Specialty S.A., LA CHAUX-DE-FONDS (CH)

(72) Inventors: Sanjib DAS, Kolkata (IN); Sachin Sundarlal CHAUDHARI, Navi Mumbai (IN); Laxmikant Atmaram GHARAT, Thane (W) (IN); Suresh Mahadev KADAM, Thane (W) (IN); Sachin GAVHANE, Thane (IN); Bipin Parsottam KANSAGRA, Ahmedabad (IN); Shailesh Shrirang PAWASE, Thane (W) (IN); Anil Subhash BHUJBAL, Thane (IN); Ulhas Digambar PATIL, Kalyan (IN); Sunil Pandurang NIRGUDE, Thane (W) (IN)

(21) Appl. No.: 18/042,988

(22) PCT Filed: Sep. 3, 2021
(86) PCT No.: PCT/IB2021/058060

§ 371 Date: Feb. 24, 2023

(30) Foreign Application Priority Data
Sep. 3, 2020 (IN) 202021037956

(51) Int. Cl. C07D 241/12

(52) U.S. Cl. CPC C07D 241/12; C07B 2200/13

(57) ABSTRACT

The present invention is directed to solid state forms of a compound which has retinoid-related orphan receptor gamma (RORy) modulator activity. Particularly the present invention relates to solid state forms of the compound (S)-2-(4-(1,1-difluoro-2-hydroxypropyl)phenyl)-N-(4-(3-(2-ethylphenyl)pyrazin-2-yl)phenyl) acetamide, processes for their preparation, pharmaceutical compositions containing the same and their use in therapy. More, particularly the present invention relates to crystalline form of the compound (S)-2-(4-(1,1-difluoro-2-hydroxypropyl)phenyl)-N-(4-(3-(2-ethylphenyl)pyrazin-2-yl)phenyl)acetamide, processes for their preparation, pharmaceutical compositions containing the same and their use in therapy.

### RELATED APPLICATIONS

This application claims the benefit of Indian Provisional Patent Application No. 202021037956 filed on Sep. 3, 2020 which is hereby incorporated by reference in its entirety.

### FIELD OF THE INVENTION

The present invention is directed to solid state forms of a compound which has retinoid-related orphan receptor gamma (RORy) modulator activity. Particularly the present invention relates to solid state forms of the compound (S)-2-(4-(1,1-difluoro-2-hydroxypropyl)phenyl)-N-(4-(3-(2-ethylphenyl)pyrazin-2-yl)phenyl)acetamide, processes for their preparation, pharmaceutical compositions containing the same and their use in therapy. More, particularly the present invention relates to crystalline form of the compound (S)-2-(4-(1,1-difluoro-2-hydroxypropyl)phenyl)-N-(4-(3-(2-ethylphenyl)pyrazin-2-yl)phenyl)acetamide, processes for their preparation, pharmaceutical compositions containing the same and their use in therapy.

### BACKGROUND OF THE INVENTION

Retinoid-related orphan receptors (RORs) are transcription factors which belong to the steroid hormone nuclear receptor super family. The ROR family consists of three members, ROR alpha (RORα), ROR beta (RORβ) and ROR gamma (RORγ), also known as NR1F1, NR1F2 and NR1F3 respectively (and each encoded by a separate gene RORA, RORB and RORC, respectively). RORs contain four principal domains shared by the majority of nuclear receptors: an N-terminal A/B domain, a DNA-binding domain, a hinge domain, and a ligand binding domain. Each ROR gene generates several isoforms which differ only in their N-terminal A/B domain. Two isoforms of RORγ, RORγ1 and RORγt (also known as RORγ2) have been identified.

RORyt is a truncated form of RORγ, lacking the first N-terminal 21 amino acids and is exclusively expressed in cells of the lymphoid lineage and embryonic lymphoid tissue inducers (Sun et al., Science, 2000, 288, 2369-2372; Eberl et al., Nat Immunol., 2004, 5: 64-73) in contrast to RORγ which is expressed in multiple tissues (heart, brain, kidney, lung, liver and muscle).

RORyt has been identified as a key regulator of Thl7 cell differentiation. Thl7 cells are a subset of T helper cells which produce IL-17 and other proinflammatory cytokines and have been shown to have key functions in several mouse autoimmune disease models including experimental autoimmune encephalomyelitis (EAE) and collagen-induced arthritis (CIA). In addition, Thl7 cells have also been associated in the pathology of a variety of human inflammatory and autoimmune disorders including multiple sclerosis, rheumatoid arthritis, psoriasis, Crohn’s disease and asthma (Jetten et al., Nucl. Recept. Signal, 2009, 7:e003; Manel et al., Nat. Immunol., 2008, 9, 641-649). The pathogenesis of chronic autoimmune diseases including multiple sclerosis and rheumatoid arthritis arises from the break in tolerance towards self-antigens and the development of auto-aggressive effector T cells infiltrating the target tissues. Studies have shown that Thl7 cells are one of the important drivers of the inflammatory process in tissue-specific autoimmunity (Steinman et al., J. Exp. Med., 2008, 205: 1517-1522; Leung et al., Cell. Mol. Immunol., 2010 7: 182-189). Th17 cells are activated during the disease process and are responsible for recruiting other inflammatory cell types, especially neutrophils, to mediate pathology in the target tissues (Korn et al., Annu. Rev. Immunol., 2009, 27:485-517) and RORyt has been shown to play a critical role in the pathogenic responses of Thl7 cells (Ivanov et al., Cell, 2006 126: 1121-1133). RORyt deficient mice have shown no Thl7 cells and also resulted in amelioration of EAE. The genetic disruption of RORγ in a mouse colitis model also prevented colitis development (Buonocore et al., Nature, 2010, 464: 1371-1375). The role of RORyt in the pathogenesis of autoimmune or inflammatory diseases has been well documented in the literature. (Jetten et al., Adv. Dev. Biol., 2006, 16:313-355; Meier et al. Immunity, 2007, 26:643-654; Aloisi et al., Nat. Rev. Immunol., 2006, 6:205-217; Jager et al., J. Immunol., 2009, 183:7169-7177; Serafmi et al., Brain Pathol., 2004, 14: 164-174; Magliozzi et al., Brain, 2007, 130: 1089-1104; Barnes et al., Nat. Rev. Immunol., 2008, 8: 183-192).

In addition, RORyt is also shown to play a crucial role in other non-Thl7 cells, such as mast cells (Hueber et al., J Immunol., 2010, 184: 3336-3340). RORyt expression and secretion of Thl7-type of cytokines has also been reported in NK T-cells (Eberl et al., Nat. Immunol., 2004, 5: 64-73) and gamma-delta T-cells (Sutton et al, Nat. Immunol., 2009, 31: 331-341; Louten et al., J Allergy Clin. Immunol., 2009, 123: 1004-1011), suggesting an important function for RORyt in these cells.

PCT patent application PCT/IB2016/054639 discloses a series of novel difluoro acetamide compounds having potent RORyt activity. In particular, the compound (S)-2-(4-(1,1-difluoro-2-hydroxypropyl)phenyl)-N-(4-(3-(2-ethylphenyl) pyrazin-2-yl)phenyl) acetamide, that is to say the compound having the structural formula

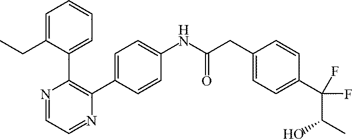 [SMILES: CCc1ccccc1-c1nccnc1-c1ccc(NC(=O)Cc2ccc(C(F)(F)[C@H](C)O)cc2)cc1]

and its preparation, is disclosed as Example 52 in the application. The PCT publication was published on Feb. 09, 2017 as publication no. WO/2017/021879, and is hereby incorporated by reference.

Development of a commercial drug candidate involves many steps, such as development of a cost effective synthetic method which is efficient in large scale manufacturing process. Also, in formulation of drug compositions, it is important for the active pharmaceutical ingredient to be in a form in which it can be conveniently handled and processed. Convenient handling is important not only from the perspective of obtaining a commercially viable manufacturing process, but also from the perspective of subsequent manufacture of pharmaceutical formulations comprising the active pharmaceutical ingredient. The drug development therefore involves research regarding finding suitable pharmaceutically acceptable salt forms of a drug. It may be also desirable to explore various polymorphs of the active pharmaceutical ingredient, which display better handling properties as well as it may also show improved physicochemical as well as pharmacokinetic and pharmacodynamics properties.

### SUMMARY OF THE INVENTION

The present application relates to novel solid state forms of compound (S)-2-(4-(1,1-difluoro-2-hydroxypropyl)phenyl)-N-(4-(3-(2-ethylphenyl) pyrazin-2-yl)phenyl) acetamide. Further the present application relates to improved process for the manufacture of compound (S)-2-(4-(1,1-difluoro-2-hydroxypropyl)phenyl)-N-(4-(3-(2-ethylphenyl) pyrazin-2-yl)phenyl) acetamide which is suitable for large scale synthesis. The process of the present application is suitable for large scale synthesis and provides the compound in high yield with high purity.

In one aspect, the present application relates to solid state form of the compound (S)-2-(4-(1,1-difluoro-2-hydroxypropyl)phenyl)-N-(4-(3-(2-ethylphenyl) pyrazin-2-yl)phenyl) acetamide represented by structural formula.

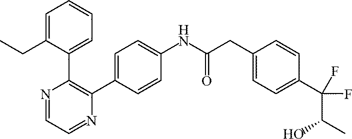 [SMILES: CCc1ccccc1-c1nccnc1-c1ccc(NC(=O)Cc2ccc(C(F)(F)[C@H](C)O)cc2)cc1]

The compound (S)-2-(4-(1,1-difluoro-2-hydroxypropyl)phenyl)-N-(4-(3-(2-ethylphenyl) pyrazin-2-yl)phenyl) acetamide, herein after, is alternatively designated as compound of formula (I).

In an embodiment, the solid state forms of compound of formula (I) exist in an anhydrous and/or solvent-free form or as a hydrate and/or a solvate form.

In another embodiment, the present application relates to (S)-2-(4-(1,1-difluoro-2-hydroxypropyl)phenyl)-N-(4-(3-(2-ethylphenyl) pyrazin-2-yl)phenyl) acetamide in crystalline form.

In another embodiment, the present application relates to (S)-2-(4-(1,1-difluoro-2-hydroxypropyl)phenyl)-N-(4-(3-(2-ethylphenyl) pyrazin-2-yl)phenyl) acetamide in amorphous form.

In another embodiment, the present application relates to a crystalline form of the compound (S)-2-(4-(1,1-difluoro-2-hydroxypropyl)phenyl)-N-(4-(3-(2-ethylphenyl) pyrazin-2-yl)phenyl) acetamide, which is designated as Form A.

In this embodiment of the present invention, the crystalline form of the compound of formula (I) is in substantially pure crystalline form. For the purpose of the present invention, the term “substantially pure” as used herein includes reference to crystalline form of, or greater than, 90%, more preferably 95%, more preferably 97%, most preferably 99% polymorphic purity as determined, for example by X-ray powder diffraction, Raman spectroscopy or IR spectroscopy.

In yet another embodiment, the present invention relates to a process for preparing the crystalline form of the compound (S)-2-(4-(1,1-difluoro-2-hydroxypropyl)phenyl)-N (4-(3-(2-ethylphenyl) pyrazin-2-yl)phenyl) acetamide, which is designated as Form A.

In yet another embodiment, the present invention relates to an amorphous form of the compound (S)-2-(4-(1,1-difluoro-2-hydroxypropyl)phenyl)-N-(4-(3-(2-ethylphenyl) pyrazin-2-yl)phenyl) acetamide.

In yet another embodiment, the present invention relates to a process for preparing the amorphous form of the compound (S)-2-(4-(1,1-difluoro-2-hydroxypropyl)phenyl)-N-(4-(3-(2-ethylphenyl) pyrazin-2-yl)phenyl) acetamide.

In yet another embodiment, the present invention relates to a pharmaceutical composition comprising the crystalline form of the compound (S)-2-(4-(1,1-difluoro-2-hydroxypropyl)phenyl)-N-(4-(3-(2-ethylphenyl) pyrazin-2-yl)phenyl) acetamide, which is designated as Form A, and one or more pharmaceutically acceptable excipient(s).

In another embodiment, there is provided a method for treating diseases, conditions and/or disorders modulated by RORyt, such as autoimmune disease, inflammatory disease, respiratory disorders, pain or cancer, comprising administering to a subject in need thereof a crystalline form of the compound (S)-2-(4-(1,1-difluoro-2-hydroxypropyl)phenyl)-N-(4-(3-(2-ethylphenyl) pyrazin-2-yl)phenyl) acetamide or a pharmaceutical composition that comprises a crystalline form of the compound (S)-2-(4-(1,1-difluoro-2-hydroxypropyl)phenyl)-N-(4-(3-(2-ethylphenyl) pyrazin-2-yl)phenyl) acetamide along with one or more pharmaceutically acceptable excipient(s).

In another aspect of the present invention, there is provided a crystalline compound of formula (I) having an average particle size value (D₅₀) in the range from about 1 µm to about 100 µm.

In another embodiment, there is provided the crystalline compound of formula (I) having an average particle size value (D₅₀) in range from about 1 µm to about 50 µm.

In yet another embodiment, there is provided crystalline compound of formula (I) having an average particle size value (D₅₀) in the range from about 1 µm to about 20 µm.

In another embodiment, there is provided crystalline compound of formula (I) having a D₁₀ value in the range from about 0.3 µm to about 10 µm.

In yet another embodiment, there is provided crystalline compound of formula (I) having a D₁₀ value in the range from about 0.3 µm to about 8 µm.

In yet another embodiment, there is provided crystalline compound of formula (I) having a D₁₀ value in the range from about 0.3 µm to about 5 µm.

In another embodiment, there is provided crystalline compound of formula (I) having a D₉₀ value in the range from about 3 µm to about 300 µm.

In yet another embodiment, there is provided crystalline compound of formula (I) having a D₉₀ value in the range from about 3 µm to about 250 µm.

In yet another embodiment, there is provided crystalline compound of formula (I) having a D₉₀ value in the range from about 3 µm to about 200 µm.

In yet another embodiment, there is provided the compound of formula (I) having a D₉₀ value in the range from about 3 µm to about 100 µm.

In another aspect of the present invention, there is provided substantially pure compound (S)-2-(4-(1,1-difluoro-2-hydroxypropyl)phenyl)-N-(4-(3-(2-ethylphenyl) pyrazin-2-yl)phenyl) acetamide (compound of formula (I)). For the purpose of the present invention, the term “substantially pure” as used herein includes reference to purity of, or greater than, 98%, more preferably 99%, more preferably 99.5%, more preferably 99.9% purity as determined, for example by HPLC.

In another embodiment, the present invention relates to substantially pure compound of formula (I) having purity greater than about 98% by HPLC.

In yet another embodiment, the present invention relates to substantially pure compound of formula (I) having purity greater than about 99% by HPLC.

In yet another embodiment, the present invention relates to substantially pure compound of formula (I) having purity greater than about 99.9% by HPLC.

In yet another aspect of the application, there is provided improved process for preparation of the compound (S)-2-(4-(1,1-difluoro-2-hydroxypropyl)phenyl)-N-(4-(3-(2-ethylphenyl) pyrazin-2-yl)phenyl) acetamide (compound of formula (I)) or its pharmaceutically acceptable salts.

In an embodiment, the present invention relates to a process for the preparation of compound of formula (I) or its pharmaceutically acceptable salt thereof, the process comprising the step of:
(a) reacting compound of formula (II)

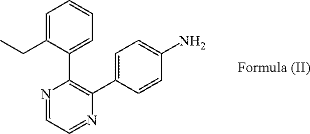 [SMILES: CCc1ccccc1-c1nccnc1-c1ccc(N)cc1]

with compound of formula (III)

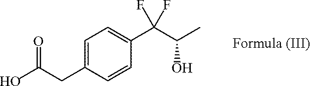 [SMILES: C[C@H](O)C(F)(F)c1ccc(CC(=O)O)cc1]

to obtain compound of formula (I) and
(b) optionally converting compound of formula (I) to its pharmaceutically acceptable salt.

In yet another embodiment, the present invention relates to a process for the preparation of compound of formula (I) or its pharmaceutically acceptable salt, the process comprising the step of:
(a) asymmetric reduction of compound of formula (IV)

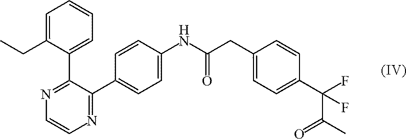 [SMILES: CCc1ccccc1-c1nccnc1-c1ccc(NC(=O)Cc2ccc(C(F)(F)C(C)=O)cc2)cc1]

using a chiral ruthenium (II) complex in presence of a hydrogen donor source; and
(b) optionally converting compound of formula (I) to its pharmaceutically acceptable salt.

In yet another aspect of the application, there is provided the compound of formula (III)

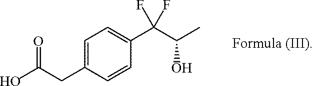 [SMILES: C[C@H](O)C(F)(F)c1ccc(CC(=O)O)cc1]

In an embodiment, the present invention relates to a process for the preparation of compound of formula (III).

### BRIEF DESCRIPTION OF THE FIGURES

FIG. 1 is powder X-ray diffraction pattern of crystalline form of (S)-2-(4-(1,1-difluoro-2-hydroxypropyl)phenyl)-N- (4-(3-(2-ethylphenyl) pyrazin-2-yl)phenyl) acetamide, designated as Form A.

FIG. 2 is Infra-Red (IR) spectra of crystalline form of (S)-2-(4-(1,1-difluoro-2-hydroxypropyl)phenyl)-N-(4-(3-(2-ethylphenyl) pyrazin-2-yl)phenyl) acetamide, designated as Form A.

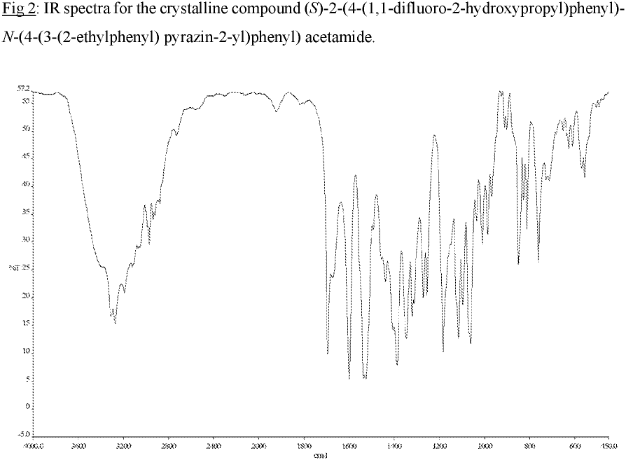

FIG. 3 is powder X-ray diffraction pattern of amorphous form of (S)-2-(4-(1,1-difluoro-2-hydroxypropyl)phenyl)-N-(4-(3-(2-ethylphenyl) pyrazin-2-yl)phenyl) acetamide.

### DETAILED DESCRIPTION OF THE INVENTION

In one aspect, the present application relates to solid state form of the compound (S)-2-(4-(1,1-difluoro-2-hydroxypropyl)phenyl)-N-(4-(3-(2-ethylphenyl) pyrazin-2-yl)phenyl) acetamide represented by structural formula

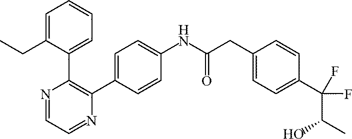 [SMILES: CCc1ccccc1-c1nccnc1-c1ccc(NC(=O)Cc2ccc(C(F)(F)[C@H](C)O)cc2)cc1]

In another embodiment, the present application relates to (S)-2-(4-(1,1-difluoro-2-hydroxypropyl)phenyl)-N-(4-(3-(2-ethylphenyl) pyrazin-2-yl)phenyl) acetamide in crystalline form.

In another embodiment, the present application relates to a crystalline form of the compound of formula (I), which is designated as Form A.

In yet another embodiment, the present invention relates to a process for preparing the crystalline form of compound of formula (I), which is designated as Form A.

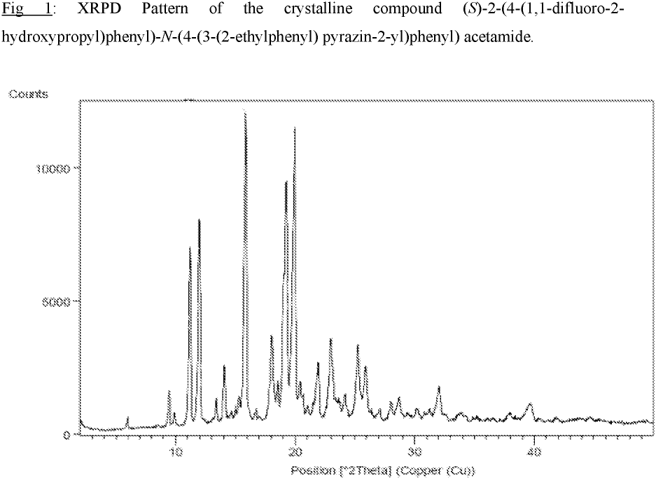

In yet another embodiment, Form A of the compound of formula (I) is characterized by the X-Ray Powder Diffraction (XRPD) pattern as shown in FIG. 1.

In yet another embodiment, Form A of the compound of formula (I) is characterized by the characteristic X-ray diffraction pattern comprising one or more of the following peaks expressed in terms of 2θ ±0.2: 12.01, 15.88 and 19.97.

In yet another embodiment, Form A of the compound of formula (I) is characterized by the characteristic X-ray diffraction pattern comprising peaks, at about 11.22, 12.01, 15.88, 18.06, 19.28 and 19.97 degrees two theta ±0.2.

In yet another embodiment, Form A of the compound of formula (I) is characterized by the characteristic X-ray diffraction pattern comprising peaks, at about 11.22, 12.01, 15.88, 18.06 and 19.97 degrees two theta ±0.2.

In yet another embodiment, Form A of the compound of formula (I) is characterized by the characteristic X-ray diffraction pattern comprising peaks, at about 11.22, 12.01, 14.1, 15.88, 18.06, 19.28, 19.97, 21.96, 22.95, 25.25 and 25.97 degrees two theta ±0.2

In yet another embodiment, Form A of the compound of formula (I) is characterized by the characteristic X-ray diffraction pattern peaks expressed in terms of 2θ as presented in Table 1.

TABLE 1

Prominent two theta positions and relative intensities of XRPD of the crystalline form of compound of formula (I)

| Angle (2θ ±0.2) | Relative intensity (%) | Angle (2θ ±0.2) | Relative intensity (%) |
|---|---|---|---|
| 9.47 | 11.72 | 19.97 (20.0) | 94.56 |
| 11.22 (11.3) | 58.09 | 20.43 | 11.81 |
| 12.01 (12.1) | 65.44 | 21.96 | 17.32 |
| 13.43 | 7.97 | 22.95 | 25.89 |
| 14.10 | 18.52 | 24.17 | 7.60 |
| 15.33 | 8.09 | 25.25 | 24.71 |
| 15.88 (15.9) | 100.00 | 25.97 | 17.12 |
| 18.06 (18.1) | 27.59 | 28.04 | 5.72 |
| 18.57 | 12.14 | 28.73 | 7.44 |
| 19.02 | 45.84 | 32.04 | 11.37 |
| 19.28 | 74.34 | 39.83 | 4.95 |

In yet another embodiment, Form A of the compound of formula (I) is characterized by having a melting point at 190° C. ± 1, done by differential scanning calorimetry (DSC).

In yet another embodiment, the present invention relates to an amorphous form of the compound (S)-2-(4-(1,1-difluoro-2-hydroxypropyl)phenyl)-N-(4-(3-(2-ethylphenyl) pyrazin-2-yl)phenyl) acetamide.

In yet another embodiment, the present invention relates to a process for preparing the amorphous form of compound (S)-2-(4-(1,1-difluoro-2-hydroxypropyl)phenyl)-N-(4-(3-(2-ethylphenyl) pyrazin-2-yl)phenyl) acetamide.

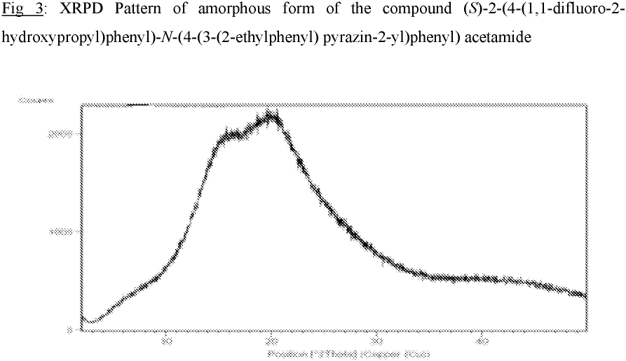

In yet another embodiment, the present application relates to an amorphous form of the compound of formula (I) which is characterized by the X-Ray Powder Diffraction (XRPD) pattern as shown in FIG. 3.

In another aspect of the present invention, there is provided substantially pure compound (S)-2-(4-(1,1-difluoro-2-hydroxypropyl)phenyl)-N-(4-(3-(2-ethylphenyl) pyrazin-2-yl)phenyl) acetamide (compound of formula (I)). For the purpose of the present invention, the term “substantially pure” as used herein includes reference to purity of, or greater than, 98%, more preferably 99%, more preferably 99.5%, more preferably 99.9% purity as determined, for example by HPLC.

In another embodiment, the present invention relates to substantially pure compound of formula (I) having purity greater than about 98% by HPLC.

In yet another embodiment, the present invention relates to substantially pure compound of formula (I) having purity greater than about 99% by HPLC.

In yet another embodiment, the present invention relates to substantially pure compound of formula (I) having purity greater than about 99.9% by HPLC.

In another embodiment, there is provided crystalline compound of formula (I) having a D₉₀ value in the range from about 3 µm to about 300 µm.

In yet another embodiment, there is provided crystalline compound of formula (I) having a D₉₀ value in the range from about 3 µm to about 250 µm.

In yet another embodiment, there is provided crystalline compound of formula (I) having a D₉₀ value in the range from about 3 µm to about 200 µm.

In yet another embodiment, there is provided the compound of formula (I) having a D₉₀ value in the range from about 3 µm to about 100 µm.

In another aspect of the present invention, there is provided a crystalline compound of formula (I) having an average particle size value (D₅₀) in the range from about 1 µm to about 100 µm.

In another embodiment, there is provided the crystalline compound of formula (I) having an average particle size value (D₅₀) in range from about 1 µm to about 50 µm.

In yet another embodiment, there is provided crystalline compound of formula (I) having an average particle size value (D₅₀) in the range from about 1 µm to about 20 µm.

In another embodiment, there is provided crystalline compound of formula (I) having a D₁₀ value in the range from about 0.3 µm to about 10 µm.

In yet another embodiment, there is provided crystalline compound of formula (I) having a D₁₀ value in the range from about 0.5 µm to about 8 µm.

In yet another embodiment, there is provided crystalline compound of formula (I) having a D₁₀ value in the range from about 0.5 µm to about 5 µm.

The particle size characteristic for compound of formula (I) for some of the batches is provided in Table 2.

TABLE 2

| Batch. No | Particle size | | |
|---|---|---|---|
| | d (0.1) µm | d (0.5) µm | d (0.9) µm |
| 1 | 2.0 | 7.1 | 33.8 |
| 2 | 1.2 | 2.1 | 3.7 |
| 3 | 0.11 | 1.4 | 3.3 |
| 4 | 1.3 | 2.2 | 4.0 |
| 5 | 1.3 | 2.2 | 3.8 |
| 6 | 1.2 | 2.1 | 3.8 |
| 7 | 0.71 | 1.7 | 3.7 |
| 8 | 0.77 | 1.6 | 3.2 |

In another aspect, the present invention relates to process for preparing the crystalline form of compound of formula (I), which is designated as Form A.

According to an embodiment of present invention there is provided a process for preparation of the crystalline Form A of (S)-2-(4-(1,1-difluoro-2-hydroxypropyl)phenyl)-N-(4-(3-(2-ethylphenyl) pyrazin-2-yl)phenyl) acetamide of compound formula (I) comprising,
i) treating (S)-2-(4-(1,1-difluoro-2-hydroxypropyl)phenyl)-N-(4-(3-(2-ethylphenyl) pyrazin-2-yl)phenyl) acetamide of compound formula (I) with a solvent;
ii) heating the reaction mass and gradually cooling; and
iii) isolating the crystalline Form A of (S)-2-(4-(1,1-difluoro-2-hydroxypropyl)phenyl)-N-(4-(3-(2-ethylphenyl) pyrazin-2-yl)phenyl) acetamide of compound formula (I).

The process to prepare Form A of compound of formula (I), comprises taking the compound of formula (I) in a solvent wherein the solvent is selected from the group consisting of ethyl acetate, methanol, ethanol, isopropanol acetonitrile, acetone, methyl acetate, acetic acid, ethylene glycol and 1,4-dioxane. The reaction mixture may be heated at an elevated temperature for a suitable period of time to get a clear solution followed by cooling the reaction mixture to a suitable lower temperature to obtain the crystalline solid compound of formula (I). The suitable temperature at which the reaction mixture is refluxed may be 80° C. to 100° C. The suitable period for which the reaction mixture is refluxed may be 10 minutes to 1 hour. The suitable temperature at which the reaction mixture is cooled may be 20° C. to 30° C. The solid obtained may be collected by methods including decantation, centrifugation, gravity filtration, suction filtration, or any other technique for the recovery of solids. In a preferred embodiment, the solid may be filtered and washed with a solvent and dried under vacuum.

The recovered solid may be optionally further dried. Drying may be carried out in a tray dryer, vacuum oven, air oven, fluidized bed drier, spin flash dryer, flash dryer, or the like. The drying may be carried out at atmospheric pressure or under a reduced pressure at suitable temperatures as long as the compound of formula (I) is not degraded in quality. The drying may be carried out for any desired time until the required purity is achieved. For example, it may vary from about 1 to about 10 hours or longer.

In another embodiment, the process to prepare Form A of compound of formula (I), comprises taking the compound of formula (I) in a solvent and heated to get a clear solution. To that clear solution, an anti-solvent is added to precipitate out the desired Form A. In an embodiment the solvent-anti solvent combination may be methyl ethyl ketone:hexane; methnaol:diisopropyl ether; methanol: hexane; acetone: methyl tert. butyl ether; acetone:hexane; THF:methyl tert. butyl ether; THF:hexane; toluene:methanol; methyl ethyl ketone:methyl tert. butyl ether; methyl ethyl ketone:diisopropyl ether; THF:diisopropyl ether; ethyl acetate:methanol and acetone:diisopropyl ether.

The recovered solid may be optionally further dried. Drying may be carried out in a tray dryer, vacuum oven, air oven, fluidized bed drier, spin flash dryer, flash dryer, or the like. The drying may be carried out at atmospheric pressure or under a reduced pressure at suitable temperatures as long as the compound of formula (I) is not degraded in quality. The drying may be carried out for any desired time until the required purity is achieved. For example, it may vary from about 1 to about 10 hours or longer.

In another embodiment, the process to prepare Form A of compound of formula (I), comprises taking the compound of formula (I) in a solvent and heated to get a clear solution. To that clear solution water is added to precipitate out the desired Form A. In an embodiment the solvent may be selected from N-methyl-2-pyrrolidone, THF, acetone, methanol, dimethylacetamide, dimethylformamide, 1-4 dioxane, and methyl ethyl ketone.

The recovered solid may be optionally further dried. Drying may be carried out in a tray dryer, vacuum oven, air oven, fluidized bed drier, spin flash dryer, flash dryer, or the like. The drying may be carried out at atmospheric pressure or under a reduced pressure at suitable temperatures as long as the compound of formula (I) is not degraded in quality. The drying may be carried out for any desired time until the required purity is achieved. For example, it may vary from about 1 to about 10 hours or longer.

In another aspect, the present invention relates to a process for preparing compound of formula (I) in amorphous form.

According to an embodiment of present invention, there is provided a process for preparation of the amorphous form of (S)-2-(4-(1,1-difluoro-2-hydroxypropyl)phenyl)-N-(4-(3-(2-ethylphenyl) pyrazin-2-yl)phenyl) acetamide of compound formula (I) comprising,
(a) heating (S)-2-(4-(1,1-difluoro-2-hydroxypropyl)phenyl)-N-(4-(3-(2-ethylphenyl) pyrazin-2-yl)phenyl) acetamide of compound formula (I) to melt under vacuum; and
(b) cooling the melted compound formula (I) obtained in step (a), so as to provide amorphous form of (S)-2-(4-(1,1-difluoro-2-hydroxypropyl)phenyl)-N-(4-(3-(2-ethylphenyl) pyrazin-2-yl)phenyl) acetamide of compound formula (I)

According to another embodiment of present invention, there is provided a process for preparing an amorphous form of (S)-2-(4-(1,1-difluoro-2-hydroxypropyl)phenyl)-N-(4-(3-(2-ethylphenyl) pyrazin-2-yl)phenyl) acetamide of compound formula (I) comprising,
(a) heating (S)-2-(4-(1,1-difluoro-2-hydroxypropyl)phenyl)-N-(4-(3-(2-ethylphenyl) pyrazin-2-yl)phenyl) acetamide of compound formula (I) to a temperature of about 150 - 300° C.; and
(b) cooling the compound formula (I) obtained in step (a) to about 20 - 50° C., so as to provide amorphous (S)-2-(4-(1,1-difluoro-2-hydroxypropyl)phenyl)-N-(4-(3-(2-ethylphenyl) pyrazin-2-yl)phenyl) acetamide of compound formula (I).

The process to prepare amorphous form of compound of formula (I), comprises taking the compound of formula (I) under vacuum in a suitable system and increasing the temperature of the system till the solid completely melts. The temperature of the system may be increased to about 150 - 300° C., preferably about 200° C. The reaction mass may be cooled to a suitable temperature to obtain the compound of formula (I) in amorphous form. The suitable temperature to which the compound may be cooled may be in the temperature range of 20-50° C., preferably 25 - 30° C.

In yet another embodiment, the present invention relates to a pharmaceutical composition comprising a therapeutically acceptable amount of a crystalline Form A of compound (S)-2-(4-(1,1-difluoro-2-hydroxypropyl)phenyl)-N-(4-(3-(2-ethylphenyl) pyrazin-2-yl)phenyl) acetamide of formula (I) and one or more pharmaceutically acceptable excipient(s).

In yet another embodiment, there is provided a method for treating diseases, conditions and/or disorders modulated by RORyt, such as autoimmune disease, inflammatory disease, respiratory disorders, pain or cancer, comprising administering to a subject in need thereof a crystalline form of compound of formula (I) designated as Form A, or a pharmaceutical composition that comprises a crystalline form of compound of formula (I) designated as Form A along with one or more pharmaceutically acceptable excipient(s).

In yet another embodiment, there is provided a method for treating diseases, conditions and/or disorders selected from chronic obstructive pulmonary disease (COPD), asthma, cough, pain, inflammatory pain, chronic pain, acute pain, arthritis, osteoarthritis, multiple sclerosis, rheumatoid arthritis, colitis, ulcerative colitis and inflammatory bowel disease, comprising administering to a subject in need thereof a crystalline form of compound of formula (I) designated as Form A, or a pharmaceutical composition that comprises a crystalline form of compound of formula (I) designated as Form A along with one or more pharmaceutically acceptable excipient(s).

In yet another embodiment, there is provided a method for treating respiratory diseases, selected from chronic obstructive pulmonary disease (COPD), asthma, bronchospasm and cough, comprising administering to a subject in need thereof a crystalline form of compound of formula (I) designated as Form A, or a pharmaceutical composition that comprises a crystalline form of compound of formula (I) designated as Form A along with one or more pharmaceutically acceptable excipient(s).

In another aspect of the application, there is provided an improved process for preparation of compound of formula (I).

In an embodiment the process to prepare compound of formula (I) comprises the step of reacting compound of formula (II) with compound of formula (III) as shown in scheme 1.

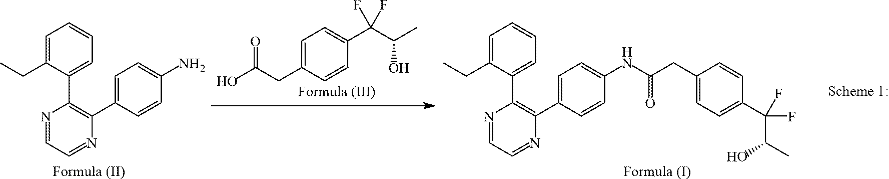

The reaction of compound of formula (II) with compound of formula (III) can be carried out using a suitable coupling agent. The suitable coupling agent used in the reaction includes, but not limited to, O-(Benzotriazol-1-yl)-N,N,N′,N′-tetramethyluronium tetrafluoroborate (TBTU), propylphosphonic anhydride (T3P), 1-[bis(dimethylamino)methylene]-1H-1,2,3-triazolo[4,5-b]pyridinium 3-oxid hexafluorophosphate) (HATU) and the like. In a preferred embodiment, the coupling agent used for the reaction is TBTU or HATU. The coupling of compound of formula (II) with compound of formula (III) can be carried out in presence of a suitable base. The suitable base used in the reaction includes, but not limited to, N, N-diisopropylethylamine (DIPEA), 4-dimethylaminopyridine (DMAP), pyridine and the like. In a preferred embodiment, the base used for the reaction is DIPEA. The reaction of compound of formula (II) with compound of formula (III) may be carried out in any polar solvent. The polar solvent used in the reaction includes, but not limited to, tetrahydofuran, ethyl acetate, acetone, dimethyl formamide, acetonitrile, dimethyl sulfoxide, methanol, ethanol, propanol, isopropanol, water and the like or mixture thereof. Preferably the solvent used is tetrahydofuran.

The compound of formula (II) is added to compound of formula (III) at a reduced temperature. The suitable temperature for such addition may be to 0-5° C. The reaction mass be stirred at a suitable temperature for a suitable period of time. The suitable temperature may be about 20-30° C. The suitable period may be 1 to 2 hour(s). After completion, the reaction may be quenched with suitable solvent such as water, ethyl acetate or mixture thereof. The reaction mixture may be extracted with a suitable solvent. The suitable solvent may be ethyl acetate. The organic layer may be dried and evaporated to dryness to obtain a product. The product obtained may be taken in another solvent and stirred for a suitable period of time. The solvent used is single solvent or a mixture of solvents wherein the solvent is selected from the group consisting of hexane, toluene, acetone, methyl ethyl ketone, tetrahydrofuran, di-isopropyl ether, methyl tert-butyl ether, acetonitrile, methanol, ethanol, isopropyl alcohol, methyl acetate, ethyl acetate, and acetic acid. Preferably, the solvent is acetonitrile. The suitable period may be 2 to 5 hours. The product may be filtered and dried using known techniques to obtain compound of formula (I). The purification step may be further repeated to obtain the desired purity.

In an embodiment, there is provided process for preparation of the crystalline form of (S)-2-(4-(1,1-difluoro-2-hydroxypropyl)phenyl)-N-(4-(3-(2-ethylphenyl)pyrazin-2-yl)phenyl) acetamide, having a characteristic X- ray diffraction pattern comprising of the peaks expressed in terms of 2θ ±0.2: 11.22, 12.01, 15.88, 18.06, 19.28 and 19.97 comprising,
(a) reacting compound of formula (II)

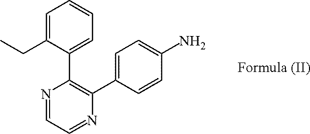 [SMILES: CCc1ccccc1-c1nccnc1-c1ccc(N)cc1]

with compound of formula (III)

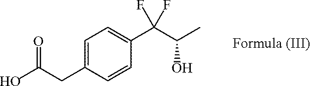 [SMILES: C[C@H](O)C(F)(F)c1ccc(CC(=O)O)cc1]

(b) treating (S)-2-(4-(1,1-difluoro-2-hydroxypropyl)phenyl)-N-(4-(3-(2-ethylphenyl) pyrazin-2-yl)phenyl) acetamide obtained in step (a) with a solvent;
(c) heating the reaction mass to get clear solution and gradually cooling and
(d) isolating the solid.

In a further embodiment, solvent is selected from acetonitrile, ethyl acetate, methanol, ethanol, isopropanol acetonitrile, acetone, methyl acetate, acetic acid, ethylene glycol and 1,4-dioxane.

In another embodiment the process to prepare compound of formula (I) comprises the asymmetric reduction of compound of formula (IV) using a chiral ruthenium (II) complex in presence of a hydrogen donor source, as depicted in scheme 2.

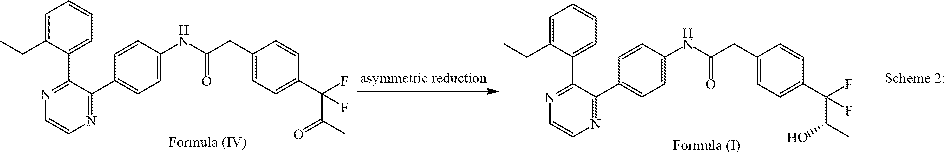

In another embodiment, the process comprises asymmetric transfer hydrogenation of compound of formula (IV) using an appropriate chiral ruthenium complexes, which may be generated in situ or used as such. The chiral ruthenium complexes used in the reaction is represented by the following formula: [RuCl(η⁶-arene)(N-arylsulfonyl-DPEN)], wherein in the complex, arene moiety may be selected from mesytelene, p-cymene, benzene and the like; aryl may be selected from p-tolyl, 1-naphthyl, 2,4,6-trimethylbenzene and the like; and DPEN is 1,2-diphenylethylenediamine.

In a preferred embodiment, the chiral ruthenium complexes may be generated in situ using dichloro(p-cymene)ruthenium(II) dimer and (1S,2S)-(+)-N-(4-toluenesulfonyl)-1,2-diphenylethylenediamine. The suitable hydrogen donor source for the reaction is isopropanol or formic acid-triethylamine. In a preferred embodiment, the suitable hydrogen donor source for the reaction is isopropanol. The reduction reaction of compound of formula (IV) may be carried out in any polar solvent such as tetrahydofuran, ethyl acetate, acetone, dimethyl formamide, acetonitrile, dimethyl sulfoxide, methanol, ethanol, propanol, isopropanol, water and the like or mixture thereof. Preferably the solvent is tetrahydofuran. The reduction reaction of compound of formula (IV) may be carried out in presence of a suitable base such as sodium tert-butoxide or potassium tert-butoxide. In a preferred embodiment, the suitable base may be potassium tert-butoxide. The asymmetric reduction of compound of formula (IV) may be carried at various suitable temperature and for suitable period of time.

In yet another aspect of the application, there is provided the compound of formula (III)

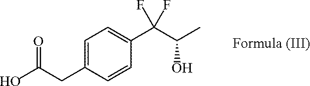 [SMILES: C[C@H](O)C(F)(F)c1ccc(CC(=O)O)cc1]

In an embodiment, the present invention relates to a process for the preparation of compound of formula (III). The compound of formula (III) can be prepared by following the process at depicted in scheme 3

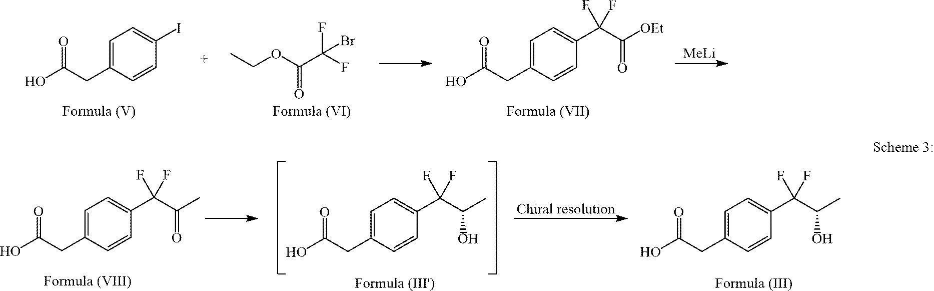

According to an embodiment, the process for the preparation of compound of formula (III)

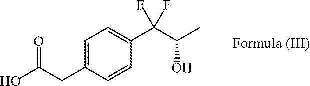 [SMILES: C[C@H](O)C(F)(F)c1ccc(CC(=O)O)cc1]

comprising,
(a) reacting compound of formula (V) with compound of formula (VI) to obtain compound of formula (VII);

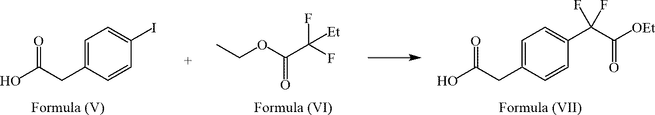

(b) reacting compound of formula (VII) with a suitable methylating agent to obtain compound of formula (VIII);

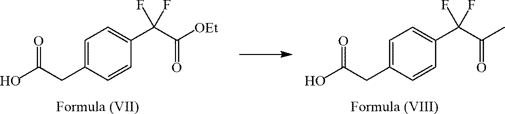

(c) reducing compound of formula (VIII) using a suitable chiral reducing agent to obtain compound of formula (III′); and

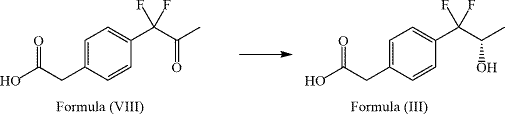

(d) optionally carrying out chiral resolution of compound of formula (III′) using a suitable amine auxiliary to obtain the compound to formula (III) with desired purity.

Step (a) involves reacting compound of formula (V) with compound of formula (VI) to obtain compound of formula (VII). The reaction can be done in presence of copper powder. The reaction can be carried out in a suitable solvent or mixture thereof. The suitable solvent may be any polar aprotic solvent such as ethyl acetate, acetone, acetonitrile, tetrahydofuran (THF), dimethyl sulfoxide (DMSO) and dimethylformamide (DMF) and the like or mixture thereof. In a preferred embodiment, the solvent may be dimethyl sulfoxide. The reaction may be carried out at suitable temperature(s) and reaction mass stirred for a suitable period of time. After completion, the reaction may be quenched with suitable solvent such as water, ethyl acetate or mixture thereof. The reaction mixture may be extracted with suitable solvent. The suitable solvent may be ethyl acetate. The organic layer may be dried and evaporated to dryness to obtain a product. The product obtained may be treated with another solvent under suitable conditions. Preferably, the solvent is methyl tert-butyl ether. The product obtained may be further treated with an amine auxiliary in a suitable solvent to enhance purity. The suitable amine auxiliary may be R-phenyl ethyl amine, methylamine, ethylamine, n-propylamine, benzylamine, tert-butyl amine, cyclopropyl amine and the like. In the preferred embodiment, the amine auxiliary may be R-(+)-Phenyl ethyl amine. The suitable solvent is selected from a dipolar aprotic organic solvent, such as an ether, for example diethyl ether, methyl tert-butyl ether, tetrahydrofuran, 2-methyltetrahydrofuran or 1,4-dioxane; a nitrile, for example acetonitrile; an amide, for example N,N-dimethylacetamide or N, N-dimethyl formamide. Preferably the reaction is carried out in an ether, such as methyl tert-butyl ether. The reaction may be stirred for a suitable period of time and dilute HCl added to the reaction mixture to adjust the pH to 2-3. The product may be filtered and dried using known techniques to obtain compound of formula (VII).

Step (b) involves reacting compound of formula (VII) with a suitable methylating agent to obtain compound of formula (VIII). The suitable methylating agent includes, but not limited to, methyl halides such as methyl iodide, methyl bromide and the like; dimethyl sulphate, methyl p-toluene sulphonate, methyl methane sulphonate, Methyl magnesium chloride, methyl lithium and the like. In a preferred embodiment, the suitable methylating agent is methyl lithium. The reaction can be carried out in a suitable solvent or mixture thereof. The suitable solvent are aliphatic hydrocarbons such as pentane, hexane, cyclohexane and petroleum ether, aromatic hydrocarbons such as toluene, o-, m- and.p-xylene, halogenated hydrocarbons such as methylene chloride, chloroform and chlorobenzene, ethers such as diethyl ether, diisopropyl ether, tert-butyl methyl ether, dioxane, anisole and tetrahydrofuran, and N-methylpyrrolidone, N-octylpyrrolidone, cyclic urea derivatives such as dimethylpropyleneurea, dimethyl sulfoxide, dimethylformamide and dimethylacetamide or mixtures thereof. In a preferred embodiment, the suitable solvent used is tert-butyl methyl ether, tetrahydrofuran or mixture thereof. After completion, the reaction may be quenched with acid solution and the reaction mixture extracted with water immiscible organic solvent. According to an embodiment the solvent used for extraction is ethyl acetate. The organic layer may be dried and evaporated to dryness to obtain a product. The product may purified using known techniques to obtain compound of formula (VIII).

Step (c) involves reducing compound of formula (VIII) using a suitable chiral reducing agent to obtain compound of formula (III′). The suitable chiral reducing agent for the reaction may be chiral oxazaborolidine reagents selected from but are not limited to (1R,2S)-cis-1-amino-2-indanol, (1S,2R)-cis-1-amino-2-indanol, (S)-prolinol, (R)-prolinol, B-(3-pinanyl)-9-borabicyclo [3.3.2] nonane (alpine-borane), 5,5-diphenyl-2-methyl-3,4-propano-1,3,2-oxazaborolidine, (S)-2-methyl-CBS-oxazaborolidine, (R)-2-methyl-CBS-oxazaborolidine. In a preferred embodiment the chiral reducing agent is (R)-2-Methyl-CBS-oxazaborolidine. The chiral reducing agent may be used along with a borane source such as borane-tetrahydrofuran (THF), borane-dimethylsulfide, borane-N,N-diethylaniline, 9-borabicyclononane (9-BBN) or diborane. In a preferred embodiment, the borane source is borane-dimethylsulfide. The reaction can be carried out in a suitable solvent or mixture thereof. The suitable solvent is an inert solvent, selected from but not limited to, toluene, tetrahydrofuran, tert-butyl methyl ether or mixtures thereof. In a preferred embodiment, the suitable solvent used is tetrahydrofuran. After completion, the reaction may be quenched with acid solution and the reaction mixture extracted with water immiscible organic solvent. According to an embodiment the solvent used for extraction is ethyl acetate. The organic layer may be dried and evaporated to dryness to obtain a product. The product may be purified using known techniques to obtain compound of formula (III′).

Step (d) involves carrying out chiral resolution of compound of formula (III′) using a suitable amine auxiliary in a suitable solvent. The suitable amine auxiliary may be selected from R-3-methyl 2-butyl amine, R-phenyl ethylamine, (R)-1-(1- naphthyl) ethylamine, (R)-1-(2-naphthyl) ethylamine, (R)-1-(4-bromophenyl) ethylamine, (R)-α-methyl-4-nitrobenzylamine, (R)-1-phenylpropylamine, (R)-1- (p-tolyl) ethylamine, (R)-1-aminoindan, (R)-1-phenyl-2-(p-tolyl)ethylamine, (R) -1-aminotetralin, (R)-3-bromo-α-methylbenzylamine, (R)-4-chloro-α- methylbenzylamine, (R)-3-methyl group-α-methylbenzylamine, (R)-2-methoxy-α-methylbenzylamine, (R)-4-methoxy-methylbenzyl-α-amine, (R)-3-amino-3-phenyl-propan-1-ol and (R)-1-amino-1-phenyl-2-methoxyethane, R- methoxyphenyl ethyl amine, R-chlorophenyl ethyl amine, R-phenyl glycinol and the like. In the preferred embodiment, the amine auxiliary is R-phenyl glycinol. The suitable solvent for the reaction is selected from but not limited to, polar aprotic solvents such as dichloromethane, acetonitrile, dimethylformamide (DMF) and the like or mixture thereof. In preferred embodiment acetonitrile is used. The amine salt obtained may be checked for purity and optionally the salt may be purified by taking it in a suitable solvent and heating the mixture till a clear solution is obtained. The temperature to which the mixture is heated may be 70-95° C. The clear solution may be stirred for a suitable period of time and then cooled to room temperature. The mixture may be allowed to stir at room temperature for suitable period of time. The solid obtained is filtered to obtain pure amine salt of compound of formula (IV). The amine salt may be taken in water and the mixture may be stirred for a suitable period of time and dilute acid added to the reaction mixture to adjust the pH to 2-3. The product obtained may be filtered and dried using known techniques to obtain compound of formula (III). The purification step may be further repeated to obtain the compound with desired purity.

According to an embodiment of present invention, the compound of formula (III) is used in preparation of compound of formula (I).

In an embodiment, the present invention relates to a process for the preparation of compound of formula (II). The compound of formula (II) can be prepared by following the process at depicted in scheme 4

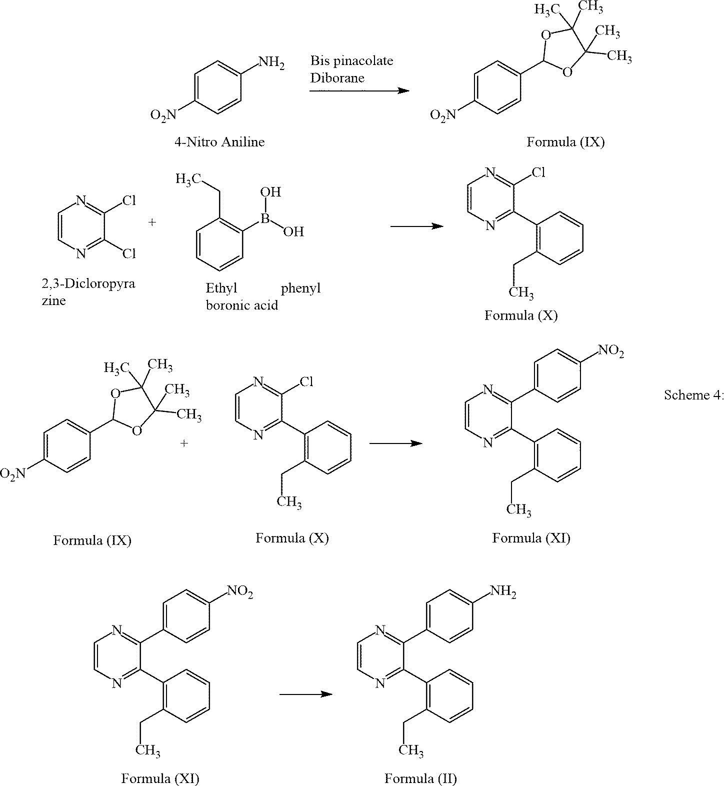

A process for preparation of compound of formula (II) comprises steps of
a) reacting 4-nitroaniline with bis pinacolate diborane to obtain compound of formula (IX)

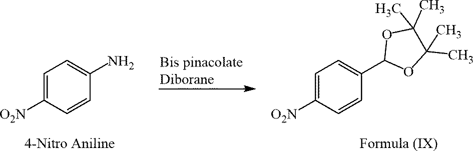

b) reacting 2,3-dichloropyrazine with ethyl phenyl boronic acid to obtain compound of formula (X)

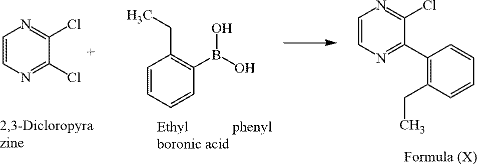

c) reacting compound of formula (IX) with compound of formula (X) to obtain compound of formula (XI)

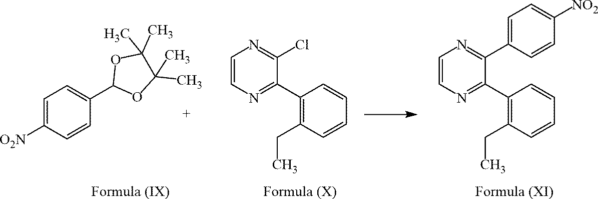

d) reducing compound of formula (XI) to obtain compound of formula (II) with desired purity.

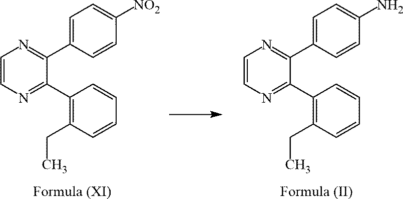

Step a) involves reacting 4-nitro aniline with bis pinacolate diborane to obtain compound of formula (IX). Said reaction is carried out in presence of an acid, a nitrite and a suitable solvent. The acid used in the reaction may be any mineral acid, in preferred embodiment HCl is used. The nitrite used is selected from alkali metal nitrite like sodium nitrite or potassium nitrite or alkyl nitrite like n-butyl nitrite. The suitable solvent used for said reaction is selected from an alcoholic solvent like methanol, ethanol, propanol, isopropanol, n-butanol, tert-butanol or mixture thereof. The reaction may be carried out at suitable temperature(s) and reaction mass stirred for a suitable period of time. After completion, the reaction may be quenched with suitable solvent such as water, ethyl acetate or mixture thereof. The reaction mixture may be extracted with suitable solvent. The suitable solvent may be ethyl acetate. The organic layer may be dried and evaporated to dryness to obtain a product. The product obtained may be treated with another solvent under suitable conditions. Preferably, the solvent is methanol.

Alternatively, Step a) involves reacting 4-nitro aniline with bis pinacolate diborane in presence of a peroxide, a nitrite and a suitable solvent.

Step b) comprises of reaction of 2,3-dichloropyrazine with ethyl phenyl boronic acid to obtain compound of formula (X). The reaction is carried out in presence of a base, a catalyst and a suitable solvent. The base used in reaction may be selected from sodium carbonate, potassium carbonate, sodium bicarbonate, potassium bicarbonate and the like. In preferred embodiment, sodium carbonate is used. The catalyst used for said reaction is preferably palladium based catalyst selected from palladium acetate, tetrakis(triphenylphosphine) palladium(0), bis(triphenylphosphine)palladium(II) dichloride, or [1,1′-bis(diphenylphosphino)ferrocene]palladium(II) dichloride and the like. The solvent used in the reaction is selected from tetrahydrofuran, 1,4-dioxane, methanol, ethanol, isopropanol, n-butyl alcohol, water or mixture thereof. The solvent is preferably 1,4-dioxane or water. The reaction may be carried out at suitable temperature(s) and reaction mass stirred for a suitable period of time. After completion, the reaction mixture is extracted with suitable organic solvent. The solvent such as ethyl acetate is used for extraction of reaction mixture. The organic layer may be dried and evaporated to dryness to obtain oily residue, which is purified by high vacuum distillation.

Step c) involves coupling of compound of formula (IX) with compound of formula (X) to obtain compound of formula (XI). Said reaction is carried out in presence of coupling catalyst, a base and a suitable solvent. The base used in reaction may be selected from sodium carbonate, potassium carbonate, sodium bicarbonate, potassium bicarbonate and the like. In preferred embodiment, sodium carbonate is used. The catalyst used for said reaction is preferably palladium based catalyst selected from palladium acetate, tetrakis(triphenylphosphine)palladium(0), bis(triphenylphosphine)palladium(II) dichloride, or [1,1′-bis(diphenylphosphino)ferrocene]palladium(II) dichloride and the like. The solvent used in the reaction is selected from tetrahydrofuran, 1,4-dioxane, methanol, ethanol, isopropanol, n-butyl alcohol, water or mixture thereof. The solvent is preferably 1,4-dioxane or water. After completion, the reaction may be quenched with suitable solvent such as water, ethyl acetate or mixture thereof. The reaction mixture may be extracted with suitable solvent. The suitable solvent may be ethyl acetate. The organic layer may be dried and evaporated to dryness to obtain an oily product. The oily product obtained may be treated with another solvent under suitable conditions. Preferably, the solvent is acetonitrile. The solution thus obtained was filtered and to the filtrate was added ethyl acetate and an acid such as HCl to precipitate product, which was filtered and dried to obtain desired product.

Step d) involves reducing compound of formula (XI) to obtain compound of formula (II) with desired purity. Preferably, the reduction of compound of formula (XI) is carried out by catalytic hydrogenation. Preferred catalysts are palladium on carbon, platinum on carbon, Raney nickel, or Raney cobalt, and the like. Preferred hydrogen sources are hydrogen gas, formic acid, ammonium formate, formate salt of alkali metal such as lithium, sodium, or potassium; hydrazine, or cyclohexene, and the like. Reaction is carried out preferably in an alcoholic solvent such as methanol, ethanol, propanol, or isopropanol. The reaction may be carried out at suitable temperature(s) and reaction mass stirred for a suitable period of time. After completion, the reaction may be quenched with suitable solvent such as water, ethyl acetate or mixture thereof. The reaction mixture may be extracted with suitable solvent. The suitable solvent may be ethyl acetate. The organic layer may be dried and evaporated to dryness to obtain a residue. The residue obtained is may be treated with another solvent under suitable conditions and is subjected to conventional workup to obtain desired product.

According to an embodiment of present invention, the compound of formula (II) is used for preparation of compound of formula (I).

### Definitions

The term “crystalline” as used herein, means having a regularly repeating arrangement of molecules or external face planes.

The term “amorphous” as used herein, means essentially without regularly repeating arrangement of molecules or external face planes.

Unless stated otherwise, percentages stated throughout this specification are weight/weight (w/w) percentages.

The term “mixture” as used herein, means a combination of at least two substances, in which one substance may be completely miscible, partially miscible or essentially immiscible in the other substance.

The term “treating” or “treatment” of a state, disorder or condition includes; (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; or (c) relieving the disease, i.e., causing regression of the state, disorder or condition or at least one of its clinical or subclinical symptoms.

The term “subject” includes mammals (especially humans) and other animals, such as domestic animals (e.g., household pets including cats and dogs) and non-domestic animals (such as wildlife).

All powder X-ray diffraction patterns were obtained using: Panalytical X′PERT-PRO diffractometer model and measured with Cu-Kα1 radiation at wavelength of 1.54060 A°. The obtained powder X-ray diffraction profiles were integrated using X′Pert High Score Plus Software.

It is meant to be understood that peak heights in a powder x-ray diffraction pattern may vary and will be dependent on variables such as the temperature, crystal size, crystal habit, sample preparation or sample height in the analysis well of the Scintag×2 Diffraction Pattern System.

All FTIR spectra were recorded using KBr on Perkin-Elmer instrument (Model: Spectrum One). The data was processed using Spectrum One Software.

As used herein, the term “average particle size” (or synonymously, “mean particle size”) refers to the distribution of particles, wherein about 50 volume percent of all the particles measured have a size less than the defined average particle size value and about 50 volume percent of all measurable particles measured have a particle size greater than the defined average particle size value. This can be identified by the term “D₅₀” or “d (0.5)”.

The term “D₁₀” refers to the distribution of particles, wherein about 10 volume percent of all the particles measured have a size less than the defined particle size value. This can be identified by the term “d(0.1)” as well. Similarly, as used herein, the term “D₉₀” refers to the distribution of particles, wherein about 90 volume percent of all the particles measured have a size less than the defined particle size value. This can be identified by the term or “d (0.9)” as well.

The average particle size can be measured using various techniques like laser diffraction, photon correlation spectroscopy and Coulter’s principle. Typically, instruments like ZETASIZER^{®} 3000 HS (Malvern^{®} Instruments Ltd., Malvern, United Kingdom), NICOMP 388™ ZLS system (PSS-Nicomp Particle Sizing Systems, Santa Barbara, CA, USA), or Coulter Counter are generally used to determine the mean particle size. Preferably, Mastersizer 2000 (Malvern^{®} Instruments Ltd., Malvern, United Kingdom) is used to determine the particle size of the particles.

### Experimental

Unless otherwise stated, work-up includes distribution of the reaction mixture between the organic and aqueous phase indicated within parentheses, separation of layers and drying the organic layer over sodium sulphate, filtration and evaporation of the solvent. Purification, unless otherwise mentioned, includes purification by silica gel chromatographic techniques, generally using ethyl acetate/petroleum ether mixture of a suitable polarity as the mobile phase. Use of a different eluent system is indicated within parentheses. The following abbreviations are used in the text: DMSO-d₆: Hexadeuterodimethyl sulfoxide; AcOEt: ethyl acetate; equiv. or eq.: equivalents; h : hour(s); L : litres; CDCl₃: deuterated chloroform; CHCl₃ : chloroform; EtOAc or EA: ethyl acetate; DCM or MDC: dichloromethane or methylene dichloride; DMSO: dimethyl sulfoxide; DMF: N,N-dimethylformamide; MTBE: Methyl tert-butyl ether; DSC: Differential scanning calorimetry; K₂CO₃ — potassium carbonate; MeOH — methanol; EtOH: ethanol; NaHCO₃: sodium bicarbonate; Na₂CO₃— sodium carbonate; THF : tetrahydrofuran; J: Coupling constant in units of Hz; RT or rt: room temperature (22-30° C.); q.s.: quantity sufficient; aq.: aqueous; equiv. or eq.: equivalents; conc. : concentrated; min: minutes; i.e. : that is; h or hrs : hours.

The parameters mentioned in the description which characterize the polymorphic nature, particle size by the measuring techniques and methods are described below:

### Particle Size Distribution Studies:

The particle size distribution was measured using Mastersizer 2000 (Malvern® instruments Ltd., Malvern, United Kingdom) with following measuring equipment and settings:

Instrument : Malvern Mastersizer 2000
Sample Handling Unit: Hydro 2000S (A)
Material R.I. : 1.59
Material absorption : 1
(Same parameters for blue light)
Dispersant Name : Water
Dispersant R.I. : 1.33
Model : General purpose
Sensitivity : Normal
Particle shape : Irregular
Measurement Time : 5.0 seconds
Background Time : 5.0 seconds
Obscuration range : 5-15 %
Stirrer speed : 1500 rpm
Measurement repeat : 3 times at zero second interval; report average (instrument averaged) results.

### Procedure:

25-50 mg of well mixed sample was transferred in a beaker. 2-3 drops of Nonidet P-40 and 4-5 drops of water was added and mixed. 25-30 mL of water was added. The mixture was sonicated for 180 seconds with continuous stirring. Sample solution in Hydro 2000S (A) accessory was added. The sample was allowed to circulate at 1500 rpm for 60 seconds and measurement was taken.

D90 and D50 values were reported.

Powder X-ray Diffraction
Name of Instrument:- X-Ray Diffractometer (PanAnalytical)
Instrumental Settings
Incident Beam Optics

| PreFIX Module | : Programmable Divergence Slit and Anti-scatter Slit |
|---|---|
| | (Offset 0.00°) |
| Filter | : Nickel |
| Soller Slit | : Soller 0.02 rad |
| Mask | : 10 mm |
| Divergence Slit | : PDS |
| | Automatic, 10 mm Irradiated length, Offset 0.00 mm |
| Anti-scatter Slit | : Slit Fixed ½° |
| Beam Knife | : Beam Knife for MPD system |
| Diffracted Beam Optics | |
| PreFIX Module | : X′Celerator (Offset 0.00°) |
| Soller Slit | : Soller 0.02 rad |
| Anti-scatter Slit | : Programmable Anti-scatter Slit |
| | Automatic, 10 mm Observed length, offset 0.00 mm |
| Detector | : X′Celerator[1] |
| | Scanning Mode, Active length (2Theta) = 2.122° |
| Measurement parameters | |
| Scan axis | : Gonio |
| Scan mode | : Continuous |
| Start angle (°) | : 2.00 |
| End angle (°) | : 50.0 |
| Step size (°) | : 0.0167 |
| Time per step (s) | : 50.0 |

### Procedure:

Adequate quantity of the sample was taken and filled in the sample holder using back-loading technique. The sample holder was loaded between the X-ray optics-path and scan using the above mentioned instrumental parameters using the X′pert collector software. The obtained powder X-ray diffraction profiles were integrated using X′Pert High Score Plus Software.

### Fourier Transform Infrared Spectroscopy (FT-IR) Studies

About 300 mg of KBr, previously dried at 200° C. and cooled, was taken into a mortar and grinded to a fine powder. 2-3 mg of the sample was added to it and was mixed well and grinded to obtain a uniform sample. A small quantity of the sample powder was taken, put it between dies and compressed it by applying 10-15 pound pressure to obtain a semi-transparent pellet. The IR spectrum of the pellet was recorded from 4000 cm⁻¹ to 450 cm⁻¹ taking air as a reference.

### HPLC (Related Substances):

Name of Instrument: High Performance Liquid Chromatography from Agilent, Shimadzu, Waters
Reagents and Solvents:
Water (Milli Q or equivalent)
Acetonitrile (Gradient grade)
Glacial Acetic acid (AR grade)
Ammonium acetate (AR grade)

### Chromatographic Conditions:

Apparatus: A High Performance Liquid Chromatograph equipped with quaternary gradient pumps, variable wavelength UV detector attached with data recorder and integrator software or equivalent.
Column: Inertsil ODS 3V, 250 mm × 4.6 mm, 5 µ
Mobile phase: A=Buffer, B= Acetonitrile (Gradient Program
Buffer: 0.01 M Ammonium acetate pH 4.0 with glacial acetic acid. Filter through 0.45µ filter paper and degas.
Diluent: Water: Acetonitrile (20:80 v/v)
Detection wavelength: UV 280 nm
Flow Rate: 1.0 mL/minute
Injection volume: 20 µL
Column temperature: 25° C.
Run time: 55 minutes
Gradient Program:

TABLE 3

| Time (minutes) | A (%) | B (%) |
|---|---|---|
| 35 | 0.01 | 65 |
| 90 | 40 | 10 |
| 35 | 45 | 65 |
| 35 | 55 | 65 |

It was observed that the sample eluted at retention time of about 21.0 minutes under these conditions.

### Preparation of Test Solution:

10 mg of the test sample was accurately weighed and transferred into a 10 mL volumetric flask.

10 mL of diluent was added and sonicated for 5 minutes to dissolve. Made up to the mark with diluent and mixed.

Similarly, blank was prepared by omitting sample.

### Procedure:

The blank was injected and then each of test solution. The responses were recoded eliminating the peaks due to blank and the chromatographic purity was calculated by area normalization method.

% Total Impurities = Sum of all the individual impurities.

Note: The highest % individual impurity should be reported as single maximum unknown impurity.

### HPLC (Enantiomeric Purity)

Name of Instrument:- High Performance Liquid Chromatography from Shimadzu Reagents and Solvents:
Diethyl amine (AR grade))
Methanol (Gradient grade)
n-Hexane (HPLC grade)
Isopropyl alcohol (HPLC grade)

### Chromatographic Conditions:

Apparatus: A High Performance Liquid Chromatograph equipped with quaternary gradient pumps, variable wavelength UV detector attached with data recorder and Integrator software or equivalent.
Column: Chiral cel OJH 250 mm×4.6 mm, 5 µ
Mobile phase A: 0.1 % Diethyl amine in [n Hexane:Isopropyl alcohol: Methanol (60:36:4) v/v]
Diluent: Isopropyl alcohol: Methanol (90:10 v/v)
Detection wavelength: UV 280 nm
Flow Rate: 0.7 mL/minute
Injection volume: 20 µL
Column temperature: 40° C.
Run time: 25 minutes

It is observed that the sample eluted at retention time of about 14.5 minutes.

Preparation of Blank solution:

Prepare blank omitting the sample.

Preparation of Reference Standard solution:

2.5 mg of the Reference Standard was accurately weighed and transferred into a 10 mL volumetric flask. 5 mL of diluent was added and sonicated for 5 minutes to dissolve. Made up to the mark with diluent and mixed.

### Preparation of Test Solution:

2.5 mg of the sample was accurately weighed and transferred into a 10 mL volumetric flask. 5 mL of diluent was added and sonicated for 5 minutes to dissolve. Made up to the mark with diluent and mixed.

### Procedure:

The blank and reference standard solution was injected and then inject sample solution. The responses were recoded eliminating all the peaks except that of sample.

The following examples are presented to provide what is believed to be the most useful and readily understood description of procedures and conceptual aspects of this invention. The examples provided below are merely illustrative of the invention and are not intended to limit the same to disclosed embodiments. Variations and changes obvious to one skilled in the art are intended to be within the scope and nature of the invention.

### EXPERIMENTAL

### Example 1: Preparation of 4-Nitrophenyl Boronic Acid Pinacol Ester

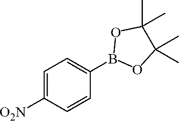 [SMILES: CC1(C)OB(c2ccc([N+](=O)[O-])cc2)OC1(C)C]

### Process- 1:

4-Nitroaniline (800 g, 5.79 mol) was taken in acetonitrile (4.8 L) in a 10 L four neck flask with nitrogen blanket. The reaction mixture was cooled to 20° C. Bis(pinacolato)diboron (1.616 Kg, 6.36 mol) and benzoyl peroxide (37.5 g, 0.1158 mol) were added to the reaction mixture and flushed with acetonitrile (850 mL). A solution of tert-butyl nitrite (716.38 g, 6.948 mol) in acetonitrile (750 mL) was slowly added to the reaction mixture maintaining the temperature between 20-35° C. The reaction mass was stirred at 20-30° C. for 3 h. After completion of the reaction, the mixture was adsorbed on 100-200 mesh silica gel. The silica was dried under vacuum at 30-35° C. for 2-3 h. The product was isolated by column chromatography in hexane-ethyl acetate. Yield: 1.08 Kg.

### Process- 2:

4-Nitroaniline (1.0 Kg, 7.24 mol) was taken in dil. HCl solution (2.5 L conc. HCl in 2.0 L of water) and methanol (5.0 L) in a 20 L four neck flask with nitrogen blanket. The reaction mixture was cooled to -5 to 0° C. A solution of sodium nitrite (748 g of sodium nitrite in 2.0 L water) was slowly added to reaction mass at 5 to 0° C., followed by addition of Bis(pinacolato)diboron (2.206 Kg, 6.36 mol). The temperature of reaction mass was then slowly raised to 20-30° C. and was maintained at said temperature for 2-3 hours. After completion of reaction, the reaction was quenched with water (8.0 L) and ethyl acetate (10.0 L) was added under stirring. The layers were separated and the aqueous layer was extracted twice with ethyl acetate. The combined organic layers were washed with water followed by saturated sodium bicarbonate solution (4.0 L). The organic layer was dried over sodium sulfate and filtered. The solvent was distilled off under vacuum and stripped with methanol (2.0 L). The residue was dried under vacuum for 2-3 h at 35-40° C. to yield desired product. Yield: 1.3 Kg ¹H NMR (400 MHz, DMSO-d₆) □ 1.166-1.321 (m, 12H), 7.902-7.924 (d, J = 8.8 Hz, 2H), 8.197-8.218 (d, J= 8.4 Hz, 2H); APCI-MS (m/z) 249.07 (M-H)^{-.}

### Example 2: Preparation of 4-[3-(2-ethylphenyl)pyrazin-2-yl]aniline

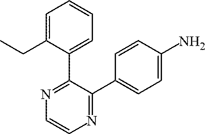 [SMILES: CCc1ccccc1-c1nccnc1-c1ccc(N)cc1]

### Process 1:

### Step (a): Preparation of 2-chloro-3-(2-ethylphenyl)pyrazine

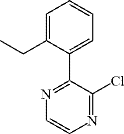

To a 3.0L four neck clean and dry flask 250 gm (1.678 mole) of 2,3-dichloropyrazine & 1.25L of 1,4-dioxane was charged under stirring. 2-Ethyl phenyl boronic acid 264.26gm (1.762 mole) was added followed by addition of 694.63 gm (5.0335 mole) of powdered potassium carbonate. Water (500 ml) was charged to reaction mass & the reaction mass was stirred for 10-15 mins at 25-30° C. Bi-triphenyl phosphine Pd (II) chloride (12.5 gm, 0.0178 mole) was slowly charged. The reaction mass was stirred for 10-15 mins & then temperature was raised to 75° C. The reaction mass was stirred and maintained for 2-3 hrs.The reaction mass was cooled to 35-40° C. & 2.0 L of ethyl acetate was charged under stirring. The reaction mass was filtered through hyflobed. The filtrate was quenched by addition of 4.0L of water under stirring. The ethyl acetate layer was separated from the aqueous layer and extracted the product from aqueous layer with ethyl acetate twice. The combined ethyl acetate layer was washed with water followed by brine solution. The organic layer was dried over sodium sulfate. The ethyl acetate layer was distilled off completely under vacuum. The residue was degassed at 40-45° C. for 1-2 hours under vacuum to give oily residue. The material was purified by silica gel column chromatography in hexane-ethyl acetate. Yield: (300.0 gm, 1.373 mol) ¹H NMR (400 MHz, DMSO-d6) d 0.963-1.00(m, 3H), 2.371-2.408 (m, 2H), 7.271-7.342 (m, 2H), 7.381-7.455 (m, 2H), 8.575-8.582(d, 2H), 8.767-8.774(d, 1H), d APCI-MS (m/z) 219.23(M-H) +.

### Step (b): Preparation of 2-(2-ethylphenyl)-3-(4-nitrophenyl)pyrazine

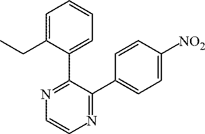 [SMILES: CCc1ccccc1-c1nccnc1-c1ccc([N+](=O)[O-])cc1]

2-chloro-3-(2-ethylphenyl)pyrazine (280 g, 1.281 mol) and 1,4-dioxane (1.12 L) were taken in a 3.0 L four neck flask under stirring. 4-Nitrophenylboronic acid pinacol ester (336.0 g, 0.1.345 mol), powdered sodium carbonate (273 g, 2.56 mol) and water (560 mL) were added to that solution. The reaction mass was stirred 10-15 min at 25-30° C. Tetrakis(triphenylphosphine)palladium(0) (45.0 g, 0.0384 mol) was slowly added to the mixture and stirred for 10-15 min. The temperature was increased to 90-100° C. and the mixture was stirred for 2-3 h. The reaction mass was cooled to 35-40° C. and ethyl acetate (3.0 L) was added under stirring. The mixture was filtered through celite. The filtrate was diluted by addition of water (4.5 L). The layers were separated and the aqueous layer was extracted twice with ethyl acetate. The combined organic layer was washed with water followed by brine. The organic layer was dried over sodium sulfate and filtered. The solvent was distilled off under vacuum and stripped with isopropyl alcohol (280 mL). The residue was dried under vacuumfor 2-3 h at 40-45° C. to yield an oily residue. The crude material was taken in isopropyl alcohol (280 mL) and stirred for 2-3 h. The solid obtained was filtered and washed with isopropyl alcohol (84 mL). The material was dried under vacuum for 6-8 h at 45° C. Yield: (260.0 gm, 0.85245 mol) ¹H NMR (400 MHz, DMSO-d₆) □ 0.898-0.935 (m, 3H), 2.308-2.364 (m, 2H), 7.169-7.232 (m, 2H), 7.293-7.311 (m, 2H), 7.354-7.371 (m, 2H), 7.589-7.611 (m, 2H), 8.127-8.149 (m, 2H), 8.824-8.848 (m, 2H); APCI-MS (m/z) 306.28 (M-H)⁺

### Step (c): Preparation of 4-[3-(2-ethylphenyl)pyrazin-2-yl]aniline

2-Ethylphenyl)-3-(4-nitrophenyl)pyrazine (250 g, 0.820 mol) and methanol (2.5 mL) were taken in a 5.0 L four neck flask under stirring. To the mixture 10% (w/w) palladium on carbon (50%wet, 25.0 gm) was added followed by addition of ammonium formate (258 g, 4.098 mol) in portions. The mixture was stirred for 10-15 min. The mixture was heated to 65° C. and stirred for 2-3 h. The reaction mass was cooled to 30-35° C. and diluted with ice-cold water (5.0 L) and ethyl acetate (2.0 L) under stirring. The mixture was filtered through celite. The layers were separated and the aqueous layer was extracted twice with ethyl acetate. The combined ethyl acetate layers were washed with water followed by brine. The organic layer was dried over sodium sulfate. It was treated with charcoal and neutral alumina under stirring. The solution was filtered through celite. The ethyl acetate was distilled off completely under vacuum followed by stripped with isopropyl alcohol (250 mL) at 45° C. The residue was dried under vacuum at 45° C. for 1-2 h to yield an oily residue. To the crude material were added isopropyl alcohol (1.0 L) followed by hydrochloric acid in isopropanol (25%, 375 mL) and the mixture was stirred for 2-3 h. The solid obtained was filtered and washed with isopropyl alcohol (125 mL). The salt obtained was dried under vacuum for 2-3 h at 45° C. The free amine was isolated by basifying the salt in water with sodium bicarbonate and the product was extracted in ethyl acetate as per regular work-up mentioned above. Yield: 155.0 gm. ¹H NMR (400 MHz, DMSO-d₆) □ 0.829-0.867 (m, 3H), 2.244-2.499 (m, 2H), 5.373 (s, 2H), 6.364-6.386 (m 2H), 7.052-7.064 (d, J= 4.8 Hz, 2H), 7.069-7.271 (m, 3H), 7.323-7.344 (m, 1H), 8.522-8.528 (d, J = 2.4 Hz, 1H), 8.626-8.632 (d, J = 2.4 Hz, 1H); APCI-MS (m/z) 276.31 (M-H)⁺

### Process 2:

### Step (a): Preparation of 2-chloro-3-(2-ethylphenyl)pyrazine

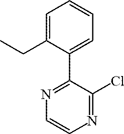

To a 3.0L four neck clean and dry flask 250 gm (1.678 mole) of 2,3-dichloropyrazine & 1.25L of 1,4-dioxane was charged under stirring. 264.26 gm (1.762 mole) of 2-ethyl phenyl boronic acid was charged followed by addition of 694.63 gm (5.0335 mole) of powdered potassium carbonate. Water (500ml) was charged to reaction mass & the reaction mass was stirred for 10-15 mins at 25-30° C. Bi-triphenyl phosphine Pd (II) chloride 12.5 gm (0.0178 mole) was charged and the reaction mass was stirred for 10-15 mins & then temperature was raised to 75° C. The reaction mass was stirred and maintained for 2-3 hrs.The reaction mass was cooled 35-40° C. & ethyl acetate (2.0 L) was charged under stirring. The reaction mass was filtered through hyflobed. The filtrate was quenched by addition of 4.0 L of water under stirring. The ethyl acetate layer was separated from the aqueous layer and extracted the product with ethyl acetate twice. The combined ethyl acetate layer was washed with water followed by brine solution. The organic layer was dried over sodium sulfate. The ethyl acetate layer was distilled off completely under vacuum. The residue was degassed at 40-45° C. for 1-2 hours under vacuum to give oily residue. The crude material was purified by silica gel column chromatography in Hexane-Ethyl acetate. Yield: (300.0 gm, 1.373 mol) 1H NMR (400 MHz, DMSO-d6) d 0.963-1.00(m, 3H), 2.371-2.408 (m, 2H), 7.271-7.342 (m, 2H), 7.381-7.455 (m, 2H), 8.575-8.582(d, 2H), 8.767-8.774(d, 1H), d APCI-MS (m/z) 219.23(M-H) +.

### Step (b): Preparation of 2-(2-ethylphenyl)-3-(4-nitrophenyl)pyrazine

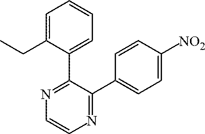 [SMILES: CCc1ccccc1-c1nccnc1-c1ccc([N+](=O)[O-])cc1]

To a 3.0L four neck clean and dry flask 280 gm (1.281 mole) of 2-chloro-3-(2-ethylphenyl)pyrazine & 1.12 L of 1,4-dioxane was charged under stirring. 336.0 gm (0.1.345 mole) 4-Nitro phenyl boronic acid pinacol ester was charged followed by addition of 530.53 gm (3.844 mole) of powdered Sodium carbonate. Water (560ml) was charged to reaction mass & the reaction mass was stirred for 10-15 mins at 25-30° C. Bi-triphenyl phosphine Pd (II) chloride 14 gm (0.0199 mole) was slowly charged. The reaction mass was stirred for 10-15 mins & then temperature was raised to ~90-100° C. The reaction mass was stirred and maintained for 2-3 hrs. The reaction mass was cooled 35-40° C. & 3.0 L of Ethyl acetate was charged under stirring. The reaction mass was filtered through hyflobed. Filtrate was quenched by addition of 4.5 L of water under stirring. The ethyl acetate layer was separated from the aqueous layer and the product was extracted with ethyl acetate twice. The combined ethyl acetate layers were washed with water followed by brine solution. The organic layer wasdried over sodium sulfate. The ethyl acetate layer was distilled off completely under vacuum followed by stripping with 280ml of Acetonitrile. The residue was degassed at 40-45° C. for 30 mins under vacuum to give oily residue. To the crude material 560ml of Acetonitrile was charged followed by 1.12 L of 10-12% Ethyl acetate. HCl. The solution was stirred for 2-3 hrs. The solid was filtered followed by addition of ~140 ml of Acetonitrile & suck dried for 30 mins. To this salt 2.8 L of water was added under stirring & Sodium bi-carbonate was slowly charged till pH 8-9. The reaction mass was stirred for 1.0 hr & the solid was filtered & dried in air oven for 6-8 hrs at 50-55° C. Yield: (292.0 gm, 0.9573 mol) 1H NMR (400 MHz, DMSO-d6) d 0.917 (m, 3H), 2.308-2.364 (m, 2H), 7.169-7.232 (m, 2H), 7.293-7.311 (m, 2H), 7.354-7.371 (m, 2H), 7.589-7.611 (m, 2H), 8.127-8.149 (m, 2H), 8.824-8.848 (m, 2H), APCI-MS (m/z) 306.28 (M-H)+.

### Step (c): Preparation of 4-[3-(2-ethylphenyl)pyrazin-2-yl]aniline

2-Ethylphenyl)-3-(4-nitrophenyl)pyrazine (1 Kg, 3.278 mol) and methanol (5.0 L) were taken in a 20.0 L four neck flask under stirring. To the mixture 10% (w/w) palladium on carbon (50% wet, 100 gm) was added followed by addition of ammonium formate (1.033 g, 16.39 mol) in portions. The mixture was stirred for 10-15 min. The mixture was heated to 65° C. and stirred for 2-3 h. The reaction mass was cooled to 30-35° C. and diluted with water (10.0 L) and ethyl acetate (8.0 L) under stirring. The mixture was filtered through celite. The layers were separated and the aqueous layer was extracted twice with ethyl acetate. The combined ethyl acetate layers were washed with water followed by brine. The organic layer was dried over sodium sulfate. It was treated with charcoal under stirring. The solution was filtered through celite. The ethyl acetate was distilled off completely under vacuum followed by addition of isopropyl alcohol (3 L) and hydrochloric acid in isopropanol (25%, 2.0 L). To the mixture was then added di-isopropyl ether (5.0 L) under stirring and maintained at 20-30° C. for 1-3 h. The solid was filtered and added to water (10 L). To this mixture sodium bicarbonate was added to obtain pH of 8-9. The product was filtered and dried at 50-55° C. to get the product. Yield: 0.85 Kg.

### Example 3: Preparation of {4-[(2S)-1, 1-difluoro-2-hydroxypropyl] Phenyl} Acetic Acid

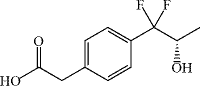 [SMILES: C[C@H](O)C(F)(F)c1ccc(CC(=O)O)cc1]

### Step (a): Preparation of [4-(2-ethoxy-1,1-difluoro-2-oxoethyl)phenyl]acetic Acid

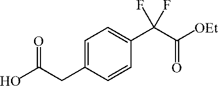 [SMILES: CCOC(=O)C(F)(F)c1ccc(CC(=O)O)cc1]

4-Iodophenylacetic acid (1.0 Kg, 3.8182 mol) was taken in dimethyl sulfoxide (6.0 L) in a 10 L four neck flask. Ethyl bromo difluoroacetate (1.55 Kg, 7.6770 mol) and copper powder (0.97 Kg, 15.3637 mol) were added to the reaction mixture at 25-30° C. under stirring. The temperature was controlled and maintained to 45° C. during the addition. The reaction mass was stirred at 25-30° C. for 10-12 h. The mixture was cooled to RT and quenched by addition of water and ethyl acetate and filtered through celite. Ammonium chloride (500 g) was added to the filtrate under stirring over a period of 10-15 min. The layers were separated and the aqueous layer was extracted twice with ethyl acetate. The combined organic layers were washed with water followed by brine. Neutral alumina (500 g) was added to the organic layer and stirred for 30-45 min followed by addition of sodium sulfate. The suspension was filtered through celite and the filtrate was distilled under vacuum at 35-40° C. followed by stripping with MTBE (500 mL). The residue was evacuated for 30-45 min at 35-40° C. MTBE (10 L) was added to the residue. The solution was cooled to 10-15° C. before R-(+)-phenyl ethyl amine (692.74 g) was slowly added over a period of 15-20 min. The mixture was stirred for 3-4 h and the solid obtained was filtered. The wet cake was transferred to a flask and stirred in water (6.0 L) at 15° C. The pH of the solution was adjusted to 2-3 by using dil. HCl. The aqueous mixture was extracted thrice with ethyl acetate. The combined organic layers were washed with water followed by brine. The organic layer was dried over anhydrous sodium sulfate and filtered. The solvent was distilled off completely under vacuum. The oily residue was stripped with hexane (500 mL) and dried under vacuum for 1-2 h. Yield: 645 gm. ¹H NMR (400 MHz, CDCl₃) □ 1.28-1.35 (m, 3H), 3.723 (s, 2H), 4.307-4.325 (d, J= 7.2 Hz, 2H), 7.393-7.413 (d, J = 8.0 Hz, 2H), 7.596-7.617 (d, J= 8.4 Hz, 2H), APCI-MS (m/z) 258.21 (M-H)^{+.}

### Step (b): Preparation of [4-(1,1-difluoro-2-oxopropyl)phenyl]acetic Acid

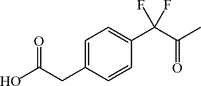 [SMILES: CC(=O)C(F)(F)c1ccc(CC(=O)O)cc1]

(2-Ethoxy-1,1-difluoro-2-oxoethyl)phenyl]acetic acid (200 g, 0.775 mol) and Methyl tert-butyl ether (2.0 L) were taken in a 10 L four neck flask with nitrogen blanket. The reaction mixture was cooled to -78° C. 1.6 M Methyl-lithium solution (970.0 mL, 1.55 mol) was added slowly maintaining the temperature between -68 to -78° C. The reaction mass was stirred at -70 to -78° C. for 30 min. The reaction was quenched by adding 10% HCl solution (~3.0 L) andstirred for another 30-45 min at 20-30° C. Ethyl acetate (2.0 L) was charged to the reaction mass and stirred for 25-30 min. The layers were separated and the aqueous layer was extracted twice with ethyl acetate. The combined ethyl acetate layer was washed with water followed by brine. The organic layer was dried over sodium sulfate, filtered and the solvent was distilled off completely under vacuum. The residue was evacuated at 35-40° C. for 1-2 h. The crude material was purified by silica gel column chromatography in hexane-ethyl acetate. Yield: (130.5 g, 0.57236 mol)

¹H NMR (400 MHz, DMSO-d₆) □□2.506 (s, 3H), 3.678 (s, 2H), 4.307-4.325 (d, J= 7.2 Hz, 2H), 7.458 (s, 2H), 7.520 (s, 2H), 12.472 (s, 1H); APCI-MS (m/z) 228.19 (M-H)^{+.}

### Step (c) Preparation of {4-[(2S)-1, 1-difluoro-2-hydroxypropyl] Phenyl} Acetic Acid

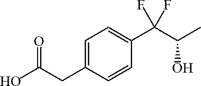 [SMILES: C[C@H](O)C(F)(F)c1ccc(CC(=O)O)cc1]

(R)-(+)methyl-CBS-oxazaborolidine (189.5 g, 0.684 mol) and tetrahydrofuran (1.56 L) were taken in a 5 L four neck flask with nitrogen blanket under stirring, The reaction mass was cooled to -5 to -10° C. Borane dimethyl sulfide complex (86.58 g, 1.14 mol) was slowly added to the reaction mass maintaining the temperature to -5 to -10° C. The reaction mass was slowly warmed up to 20-30° C. and stirred for 45-60 min. Meanwhile, a solution of [4-(1,1-difluoro-2-oxopropyl)phenyl]acetic acid (260 g, 1.140 mol) in tetrahydrofuran (520 mL) at 25-30° C. was prepared. The solution was added slowly to the above reaction mass at -5 to -10° C. The mixture was stirred for 25-30 min at the same temperature. The reaction was quenched by addition of 10% HCl solution till pH 2-3 and stirred for another 20-30 min at 20-30° C. Ethyl acetate (1.56 L) and water (1.56 L) were charged to the reaction mass and stirred for 25-30 min. The mixture was filtered through celite and the bed was washed with ethyl acetate. The layers were separated and the aqueous layer was extracted with ethyl acetate twice. The combined ethyl acetate layers were washed with water followed by brine. The organic layer was dried over sodium sulfate. The ethyl acetate layer was distilled off completely under vacuum and the residue was dried under vacuum at 35-40° C. for 1-2 h to yield an oily residue (~300 g). To this residue, were added acetonitrile (1.56 L) followed by R-3-methyl 2-butylamine (109.13 gm, 1.25438 mole) at 20-30° C. The reaction mixture was stirred for 45-60 min at 20-30° C. and then the temperature was raised to reflux temperature 80-85° C. A clear solution was observed. The solution was slowly cooled to RT and stirred for 5-6 h. The solid obtained was filtered and washed with acetonitrile.

### Purification:

In a 4-neck RBF were added the above salt and acetonitrile (8 Vol.). The solution was heated to 80-85° C. and water (0.15 vol.) was added. The clear solution was stirred for 15-20 min and slowly cooled to RT. The solution was allowed to stir at RT for 5-6 h and the solid was filtered. The material was dried under vacuum for 5-6 h at 45° C. Yield: (163 g, 0.7087 mol) ¹H NMR (400 MHz, CDCl₃)□□ 1.243-1.259 (m, 3H), 2.069 (s, 1H), 3.711 (s, 2H), 4.136-4.178 (m, 1H), 7.382-7.402 (d, J= 8.0 Hz, 2H), 7.489-7.510 (d, J= 8.4 Hz, 2H); APCI-MS (m/z) 229.27 (M-H)^{-.}

### Example 4: Preparation of {4-[(2S)-1, 1-difluoro-2-hydroxypropyl] Phenyl} Acetic Acid

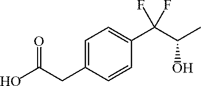 [SMILES: C[C@H](O)C(F)(F)c1ccc(CC(=O)O)cc1]

### Step (a): Preparation of [4-(1,1-difluoro-2-oxopropyl)phenyl]acetic Acid

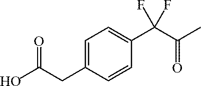 [SMILES: CC(=O)C(F)(F)c1ccc(CC(=O)O)cc1]

(2-Ethoxy-1,1-difluoro-2-oxoethyl)phenyl]acetic acid (1 kg, 3.86 mol) and tert-butyl methyl ether (8.0 L) were taken in a 20 L round bottom flask. The reaction mixture was cooled to -78° C. 1.6 M Methyl-lithium solution (6.0 L, 9.6 mol) was added slowly maintaining the temperature between -68 to -78° C. The reaction mass was stirred at -70 to -78° C. for 60 min. The reaction was quenched by adding 10% HCl solution (~15.0 L) and stirred at 20-30° C. Ethyl acetate (10.0 L) was charged to the reaction mass and stirred for 20-30 min. The layers were separated and the aqueous layer was extracted twice with ethyl acetate. The combined ethyl acetate layers were washed with water followed by brine. The organic layer was dried over sodium sulfate, filtered and the solvent was distilled off completely under vacuum. The residue was treated with 2.0 L hexane, degassed, cooled and filtered to obtain product. The product was dried at 35-40° C. for 1-2 h.

Yield: 0.80 Kg.

### Step (b) Preparation of {4-[(2S)-1, 1-difluoro-2-hydroxypropyl] Phenyl} Acetic Acid

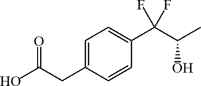 [SMILES: C[C@H](O)C(F)(F)c1ccc(CC(=O)O)cc1]

To a 5 L four neck flask with nitrogen blanketing, 1.56 L of Tetrahydrofuran and 189.5 gm (0.684 mole) of R-Methyl CBS was charged to the reaction flask under stirring. Thereaction mass was cooled to -5 to -10° C. 86.58 gm (1.14 mole) Borane-DMS was slowly charged to the reaction mass maintaining the temperature. The reaction mass was stirred at 20-30° C. for 45-60 mins. Meanwhile a solution of [4-(1,1-difluoro-2-oxopropyl)phenyl]acetic acid in Tetrahydrofuran was prepared by dissolving 260 gm (1.140 mole) in 520 ml of Tetrahydrofuran at 25-30° C. The above reaction mass was cooled to -5 to -10° C. The above prepared solution of [4-(1, 1-difluoro-2-oxopropyl) phenyl] acetic acid was slowly added to reaction mass maintaining temperature. The reaction mass was stirred & maintained for 25-30 mins. Meanwhile aq. solution of Sulfuric acid & Hydrogen peroxide (26.0 ml of Sulfuric acid & 65 ml of 50% aq. Hydrogen peroxide) in 2.6 L of water was prepared. The reaction mass was quenched by addition of this solution and stirred for 20-30 min at 20-30° C. 1.56 L of ethyl acetate and water was charged to the reaction mass and it was stirred for 25-30 minutes. The reaction mass was filtered through hyflobed followed by ethyl acetate wash. The ethyl acetate layer was separated from the aqueous layer and the product was extracted with ethyl acetate twice. The combined ethyl acetate layers were washed with water followed by brine solution. The organic layer was dried over sodium sulfate. The ethyl acetate layer was distilled off completely under vacuum. The residue was degassed at 35-40° C. for 1-2 hours under vacuum to give oily residue (~300 gm). To this oil, 1.56 L of Acetonitrile was charged followed by addition of 171.85 gm of R-(-)-2-phenyl glycinol (1.254 mole) at 20-30° C. The reaction mass was stirred for 45-60 mins and the temperature was raised to reflux temperature 80-85° C. Water (~1.2 to 1.4 v/w of input) was added to the reaction mass till clear solution observed. The reaction mass was slowly cooled to RT & it was stirred for 5-6 hrs. The solid was filtered followed by Acetonitrile wash.

The obtained solid was taken in water (10 L) and stirred at 20-30° C. for about 30 minutes. The pH of the mixture was adjusted to 3-4 by the addition of conc. HCl. Ethyl acetate (10L) was added to the reaction mixture. The ethyl acetate layer was separated from the aqueous layer and the product was extracted with ethyl acetate twice. The combined ethyl acetate layers were washed with water followed by brine solution. The organic layer was dried over sodium sulfate. The ethyl acetate layer was distilled off completely under vacuum. The residue was degassed at 35-40° C. for 1-2 hours under vacuum to give the desired product as solid. The obtained solid is optionally purified using acetonitrile. In a 4-neck RBF Above salt & 8 Volumes of Acetonitrile was charged. The temperature was raised to 80-85° C. & 1.8 to 2.1 volume of water was added. The solution was stirred for 15-20 mins & slowly cooled to RT. The stirring was continued for 5-6 hrs. The solid was filtered. The wet cake was dried under vacuum at 45° C. for 5-6 hrs. Yield: (163 g, 0.7087 mol); 1H NMR (400 MHz, CDCl3) d 1.263 (m, 3H), 2.069 (s, 1H), 3.711 (s, 2H), 4.154 (m, 1H), 7.284-7.373 (d, 2H), 7.482-7.503 (d, 2H), APCI-MS (m/z) 229.27 (M-H)^{-.} {4-[(2S)-1, 1-difluoro-2-hydroxypropyl] phenyl} acetic acid.R-3-methyl 2-butyl amine Salt ¹H NMR (400 MHz, DMSO-d₆) □ 0.831-0.848 (m, 6H), 1.010-1.075 (m, 6H), 1.658-1.707 (m, 1H), 2.804-2.831 (m, 1H), 4.005-4.076 (m, 1H), 7.292-7.360 (m, 4H). {4-[(2S)-1, 1-difluoro-2-hydroxypropyl] phenyl} acetic acid. R-(-)-phenyl-2-glycinol Salt ¹H NMR (400 MHz, DMSO) d 1.067-1.083 (m, 3H), 3.414-3.558 (m, 4H), 4.019-4.030 (d, 2H), 5.810-5.825 (bs, 4H), 7.269-7.404 (m, 9H).

### Example 5: Preparation of (S)-2-(4-(1,1-difluoro-2-hydroxypropyl)phenyl)-N-(4-(3-(2-ethylphenyl) pyrazin-2-yl) phenyl)acetamide (Process 1)

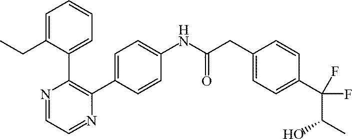 [SMILES: CCc1ccccc1-c1nccnc1-c1ccc(NC(=O)Cc2ccc(C(F)(F)[C@H](C)O)cc2)cc1]

{4-[(2S)-1, 1-difluoro-2-hydroxypropyl] phenyl}acetic acid (130 g, 0.56522 mol) and tetrahydrofuran (1.04 L) were taken in a 2.0 L four neck flask under stirring. The mixture was cooled to 0-5° C. HATU (3221.17 g, 0.84782 mol) was added to the reaction mixture and stirred for 10-15 min, followed by addition of 4-[3-(2-ethylphenyl)pyrazin-2-yl]aniline (155.4 g, 0.56522 mol). Diisopropylethylamine (145.82 g, 1.1303 mol) was added slowly and the mixture was stirred for 10-15 min at 0-5° C. The reaction mixture was warmed to 20-30° C. and stirred for another 2-3 h. The reaction mixture was quenched with water (3.0 L) and ethyl acetate was added to the reaction mixture (1.3 L) under stirring. The layers were separated and the aqueous layer was extracted twice with ethyl acetate. The combined ethyl acetate layer was washed with water and brine. The organic layer was dried over sodium sulfate. The organic layer was treated with charcoal (5% w/w) under stirring for 20-30 min. The suspension was filtered through celite. Ethyl acetate was distilled off completely under vacuum and stripped with acetonitrile (260 mL) at 45° C. The residue obtained was dried under vacuum at 45° C. for 1 h. To the crude material acetonitrile (260 mL) was added and the solution was stirred for 2-3 h. The solid obtained was filtered and dried under vacuum at 45° C. for 2-3 h. Yield: 192 gm.

### Purification:

The material, obtained above, and acetonitrile (4.6 vol.) were taken in a clean, 4-neck flask at 25-30° C. The temperature was increased to 85° C. and the reaction mass was stirred till clear solution was observed. The reaction mass was stirred for 15-20 min at the same temperature and then cooled to 20-30° C. The stirring was continued for 2-3 h at 25-30° C. The solid obtained was filtered and washed with acetonitrile (0.25 vol.). The wet cake was dried under vacuum at 45-50° C. for 5-6 h to get desired solid as crystalline Form A. Yield: 167 gm.

### Micronization:

The above material was micronized twice by air jet-mill with following parameters,
Grinding Pressure: 5-6 Kg
Ventury Pressure: 2.5-3.0 Kg
Yield: 158 gm.

¹H NMR (400 MHz, DMSO-d₆) □ 0.841-0.859 (m, 3H), 1.063-1.079 (d, J = 6.40 Hz, 2H), 2.264-2.283 (m, 2H), 3.683 (s, 2H), 4.033-4.057 (m, 1H), 5.497-5.512 (d, J = 6.0 Hz, 2H), 7.164-7.491 (m, 8H), 8.670-8.676 (d, J= 2.4 Hz, 1H), 8.733-8.739 (d, J= 2.4 Hz, 1H), 10.282 (s, 1H); APCI-MS (m/z) 488 (M-H)^{+.}

### Example 6: Preparation of (S)-2-(4-(1,1-difluoro-2-hydroxypropyl)phenyl)-N-(4-(3-(2-ethylphenyl) pyrazin-2-yl) phenyl)acetamide (Process 2)

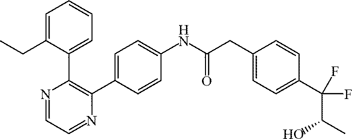 [SMILES: CCc1ccccc1-c1nccnc1-c1ccc(NC(=O)Cc2ccc(C(F)(F)[C@H](C)O)cc2)cc1]

{4-[(2S)-1, 1-difluoro-2-hydroxypropyl] phenyl}acetic acid (130 g, 0.56522 mol) and Tetrahydrofuran (1.3 L) were taken in a 2.0 L four neck flask under stirring. The mixture was cooled to -5 to 0° C. 4-[3-(2-Ethylphenyl)pyrazin-2-yl]aniline (147.68 g, 0.53635 mol) was added slowly followed by Di-isopropyl ethylamine (182.13 g, 1.4118 mol) and the mixture was stirred for 10-15 min at -5 to 0° C. TBTU (271.83 g, 0.8468 mol) was added to reaction mass in 10 equivalent lots maintaining temperature at -5 to 0° C. The reaction mixture was warmed to 20-30° C. and stirred for another 3.0 hrs. Methanol (0.325 L) & water (0.325 L) was charged to the reaction mass followed by Lithium hydroxide (70.98 g, 1.69 mol) & it was stirred for 6.0 hrs. The reaction mixture was quenched with water (1.04 L) and ethyl acetate (1.3 L) under stirring. The layers were separated and the aqueous layer was extracted twice with ethyl acetate. The combined ethyl acetate layer was washed with water and brine. The organic layer was dried over sodium sulfate. The organic layer was treated with charcoal (5% w/w) under stirring for 20-30 min. The suspension was filtered through celite. Ethyl acetate was distilled off completely under vacuum and stripped with acetonitrile (260 mL) at 45° C. The residue obtained was dried under vacuum at 45° C. for 1 h. To the material acetonitrile (260 mL) wasadded and the solution was stirred for 2-3 h. The solid obtained was filtered and dried under vacuum at 45° C. for 2-3 h. Yield: (210 g, 0.4307 mol)

### Purification:

The material, obtained above, and acetonitrile (~4.6 vol.) were taken in a clean, 4-neck flask at 25-30° C. The temperature was increased to 85° C. and it was stirred till clear to hazy solution was observed. The reaction mass was stirred for 15-20 min at the same temperature and then it was cooled to 20-30° C. The stirring was continued for 2-3 h at 25-30° C. The solid obtained was filtered and washed with acetonitrile (0.5 vol.). The wet cake was dried under vacuum at 45-50° C. for 5-6 h to get desired solid as crystalline Form A. Yield: (180 gm, 0.3692 mol)

### Micronization:

The above material was micronized twice by air jet-mill with following parameters,
Grinding Pressure: 5-6 Kg
Ventury Pressure: 2.5-3.0 Kg
Yield: (162 g, 0.3322 mol)

¹H NMR (400 MHz, DMSO-d₆) □ 0.841-0.859 (m, 3H), 1.063-1.079 (d, J = 6.40 Hz, 2H), 2.264-2.283 (m, 2H), 3.683 (s, 2H), 4.033-4.057 (m, 1H), 5.497-5.512 (d, J = 6.0 Hz, 2H), 7.164-7.491 (m, 8H), 8.670-8.676 (d, J= 2.4 Hz, 1H), 8.733-8.739 (d, J= 2.4 Hz, 1H), 10.282 (s, 1H); APCI-MS (m/z) 488 (M-H)⁺

### Example 7: Preparation of (S)-2-(4-(1,1-difluoro-2-hydroxypropyl)phenyl)-N-(4-(3-(2-ethylphenyl)pyrazin-2-yl)phenyl)acetamide (Process 3)

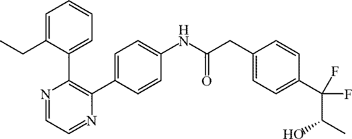 [SMILES: CCc1ccccc1-c1nccnc1-c1ccc(NC(=O)Cc2ccc(C(F)(F)[C@H](C)O)cc2)cc1]

An oven-dried flask was charged with dichloro(p-cymene)ruthenium(II) dimer (12.6 mg, 0.021 mmol), (1S,2S)-(+)-N-(4-toluenesulfonyl)-1,2-diphenylethylenediamine (15 mg, 0.041 mmol) and powdered molecular sieves (4 Å, 50 mg) at ambient temperature. To that mixture, degassed IPA (10 mL) was added and the resultant suspension was evacuated and flushed with nitrogen. The mixture was heated to 90° C. for 30 min followed by 15 min at 50° C. A solution of (2-(4-(1,1-difluoro-2-oxopropyl)phenyl)-N-(4-(3-(2-ethylphenyl)pyrazin-2-yl)phenyl)acetamide (200 mg, 0.412 mmol) in a mixture of THF and IPA (1:1, 4.0 mL) was dropwise added to the reaction mixture over a period of 10 min. After 5 min, a solution ofpotassium tert-butoxide (11.5 mg, 0.103 mmol) in IPA (2.0 mL) was slowly added to the mixture and the mixture was stirred for 2 h at 50° C. The mixture was cooled to RT and diluted with ethyl acetate (20 mL). The suspension was filtered through silica gel bed and the bed was rinsed thoroughly with ethyl acetate. The combined filtrate and washings were concentrated under reduced pressure. The residue thus obtained was purified by flash column chromatography to yield 130 mg of the desired product as a solid; ¹H NMR (300 MHz, DMSO-d₆): δ 0.85 (t, J= 7.2 Hz, 3H), 1.05 (d, J= 6.3 Hz, 3H), 2.26 (q, J= 7.2 Hz, 2H), 3.67 (s, 2H), 4.02-4.05 (m, 1H), 5.49 (d, J= 6.0 Hz, 1H), 7.16-7.34 (m, 6H), 7.39-7.49 (m, 6H), 8.66 (s, 1H), 8.72 (s, 1H), 10.27 (s, 1H); APCI-MS (m/z) 488 (M+H)⁺; Chiral HPLC purity: 99.26%.

### Example 8: Preparation of Crystalline Form A of (S)-2-(4-(1,1-difluoro-2-hydroxypropyl)phenyl)-N-(4-(3-(2-ethylphenyl)pyrazin-2-yl)phenyl)acetamide (Single solvent crystallization method)

A suspension of (S)-2-(4-(1,1-difluoro-2-hydroxypropyl)phenyl)-N-(4-(3-(2-ethylphenyl)pyrazin-2-yl)phenyl)acetamide (25.7 g, 52.77 mmol) in acetonitrile (190 mL) was refluxed for 50 min. The solution was cooled to RT under stirring and continued to stir for 18 hr at RT. The solid precipitated was filtered, washed with acetonitrile (10 mL) and dried under vacuum. The solid was further dried in air oven at 50° C. for 1 h to yield 23.37 g of the title compound in desired crystalline form. Melting temperature: 190° C. (by Differential Scanning Calorimetry).

The similar process was performed using different solvents as mentioned in the below table 4.

TABLE 4

Single Solvent Crystallization Method

| No. | Solvent | Crystalline Form |
|---|---|---|
| 1 | Ethyl acetate | Form A |
| 2 | Methanol | Form A |
| 3 | Ethanol | Form A |
| 4 | Isopropanol | Form A |
| 5 | Acetone | Form A |
| 6 | Methyl acetate | Form A |
| 7 | Acetic acid | Form A |
| 8 | Ethylene glycol | Form A |
| 9 | 1,4-Dioxane | Form A |

### Example 9: Preparation of Crystalline Form A of (S)-2-(4-(1,1-difluoro-2-hydroxypropyl)phenyl)-N-(4-(3-(2-ethylphenyl)pyrazin-2-yl)phenyl)acetamide (Binary solvent crystallization method)

(S)(4-(1,1-difluorohydroxypropyl)phenyl)-N(4-(3-(2-ethylphenyl)pyrazinyl)phenyl)acetamide was taken in methyl ethyl ketone in a suitable amount and the mixture was heated to get a clear solution. To the clear solution, hexane (an anti-solvent) was added in a suitable amount to precipitate out the solid.

The solid precipitated was filtered, and dried under vacuum.

The similar process was performed using different solvent/anti-solvent combination as mentioned in the below table 5.

TABLE 5

Binary solvent crystallization method

| No. | Solvent | Anti-Solvent | Crystalline Form |
|---|---|---|---|
| 1 | Methnaol | Diisopropyl ether | Form A |
| 2 | Methanol | Hexane | Form A |
| 3 | Acetone | Methyl tert. butyl ether | Form A |
| 4 | Acetone | Hexane | Form A |
| 5 | THF | Methyl tert. butyl ether | Form A |
| 6 | THF | Hexane | Form A |
| 7 | Toluene | Methanol | Form A |
| 8 | Methyl ethyl ketone | Methyl tert. butyl ether | Form A |
| 9 | Methyl ethyl ketone | Diisopropyl ether | Form A |
| 10 | THF | Diisopropyl ether | Form A |
| 11 | Ethyl acetate | Methanol | Form A |
| 12 | Acetone | Diisopropyl ether | Form A |

### Example 10: Preparation of Crystalline Form A of (S)-2-(4-(1,1-difluoro-2-hydroxypropyl)phenyl)-N-(4-(3-(2-ethylphenyl)pyrazin-2-yl)phenyl)acetamide (Precipitation in water method)

(S)(4-(1,1-difluorohy droxypropyl)phenyl)-N-(4-(3-(2-ethylphenyl)pyrazinyl)phenyl)acetamide was taken in N-methylpyrrolidone in a suitable amount and the mixture was heated to get a clear solution. To the clear solution, water was added in a suitable amount to precipitate out the solid. The solid precipitated was filtered, and dried under vacuum. The similar process was performed using different solvent as mentioned in the below table 6.

TABLE 6

Precipitation in water method

| No. | Solvent | Crystalline Form |
|---|---|---|
| 1 | THF | Form A |
| 2 | Acetone | Form A |
| 3 | Methanol | Form A |
| 4 | Dimethylacetamide | Form A |
| 5 | Dimethylformamide, | Form A |
| 6 | 1-4 Dioxane | Form A |
| 7 | Methyl ethyl ketone | Form A |

### Example 11: Amorphous Form of (S)-2-(4-(1,1-difluoro-2-hydroxypropyl)phenyl)-N-(4-(3-(2-ethylphenyl)pyrazin-2-yl)phenyl)acetamide

To a clean and dry RBF was charged crystalline (S)-2-(4-(1,1-difluoro-2-hydroxypropyl)phenyl)-N-(4-(3-(2-ethylphenyl)pyrazin-2-yl)phenyl)acetamide (1.0 g). The temperature was increased to ~210° C. The reaction mass was cooled and the residue was unloaded to obtain (S)-2-(4-(1,1-difluoro-2-hydroxypropyl)phenyl)-N-(4-(3-(2-ethylphenyl)pyrazin-2-yl)phenyl)acetamide in amorphous form. Melting temperature: Endotherm at 117.1° C. and Exotherm at 191.49° C. (by Differential Scanning Calorimetry).

We claim:

1( S)-2-(4-(1,1-difluoro-2-hydroxypropyl)phenyl)-N-(4-(3-(2-ethylphenyl) pyrazin-2-yl)phenyl) acetamide represented by formula (I) in crystalline form

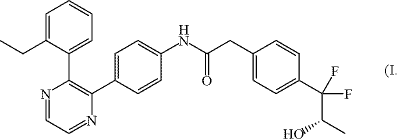 [SMILES: CCc1ccccc1-c1nccnc1-c1ccc(NC(=O)Cc2ccc(C(F)(F)[C@H](C)O)cc2)cc1]

2. A crystalline form of (S)-2-(4-(1,1-difluoro-2-hydroxypropyl)phenyl)-N-(4-(3-(2-ethylphenyl) pyrazin-2-yl)phenyl) acetamide, having a characteristic X- ray diffraction pattern comprising of the peaks expressed in terms of 2θ ±0.2: 11.22, 12.01, 15.88, 18.06, 19.28 and 19.97.

3. The crystalline form according to claim 2 having a characteristic X- ray diffraction pattern comprising of the peaks expressed in terms of 2θ ±0.2: 11.22, 12.01, 15.88, 18.06 and 19.97.

4. The crystalline compound of formula (I) according to claim 2, having an average particle size value (D₅₀) in the range from about 1 µm to about 20 µm.

5. The crystalline compound of formula (I) according to claim 2, having an average particle size value (D₅₀) in the range from about 1 µm to about 10 µm.

6. The crystalline compound of formula (I) according to claim 2, having D₉₀ value in the range from about 3 µm to about 100 µm.

7. The crystalline compound of formula (I) according to claim 2, having D₉₀ value in the range from about 3 µm to about 50 µm.

8. The crystalline compound of formula (I) according to claim 2, having purity greater than about 99% by HPLC.

9. The crystalline compound of formula (I) according to claim 2, is in substantially pure crystalline form.

10. S-2-(4-(1,1-difluoro-2-hydroxypropyl)phenyl)-N-(4-(3-(2-ethylphenyl) pyrazin-2-yl)phenyl) acetamide in amorphous form.

11. A process for preparation of the crystalline form of (S)-2-(4-(1,1-difluoro-2-hydroxypropyl)phenyl)-N-(4-(3-(2-ethylphenyl)pyrazin-2-yl)phenyl) acetamide, having a characteristic X- ray diffraction pattern comprising of the peaks expressed in terms of 2θ ±0.2: 11.22, 12.01, 15.88, 18.06, 19.28 and 19.97 comprising,
i) treating (S)-2-(4-(1,1-difluoro-2-hydroxypropyl)phenyl)-N-(4-(3-(2-ethylphenyl) pyrazin-2-yl)phenyl) acetamide of compound formula (I) with a solvent;
ii) heating the reaction mass to get clear solution and gradually cooling and
iii) isolating the solid.

12. The process according to claim 11, wherein a solvent is selected from the group consisting of acetonitrile, ethyl acetate, methanol, ethanol, isopropanol acetonitrile, acetone, methyl acetate, acetic acid, ethylene glycol and 1,4-dioxane.

13. A process for preparation of the crystalline form of (S)-2-(4-(1,1-difluoro-2-hydroxypropyl)phenyl)-N-(4-(3-(2-ethylphenyl) pyrazin-2-yl)phenyl) acetamide, having a characteristic X- ray diffraction pattern comprising of the peaks expressed in terms of 2θ ±0.2: 11.22, 12.01, 15.88, 18.06, 19.28 and 19.97 comprising,
i) treating (S)-2-(4-(1,1-difluoro-2-hydroxypropyl)phenyl)-N-(4-(3-(2-ethylphenyl) pyrazin-2-yl)phenyl) acetamide of compound formula (I) with a solvent to get a clear solution;
ii) adding an anti-solvent and
iii) isolating the solid.

14. The process according to claim 13, wherein a solvent is selected from the group consisting of acetonitrile, methanol, acetone, THF, toluene, Methyl ethyl ketone, and ethyl acetate.

15. The process according to claim 13, wherein an anti-solvent is selected from the group consisting of diisopropyl ether, hexane, methyl tert. butyl ether and methanol.

16. A process for preparation of the crystalline form of (S)-2-(4-(1,1-difluoro-2-hydroxypropyl)phenyl)-N-(4-(3-(2-ethylphenyl) pyrazin-2-yl)phenyl) acetamide, having a characteristic X- ray diffraction pattern comprising of the peaks expressed in terms of 2θ ±0.2: 11.22, 12.01, 15.88, 18.06, 19.28 and 19.97 comprising,
i) treating (S)-2-(4-(1,1-difluoro-2-hydroxypropyl)phenyl)-N-(4-(3-(2-ethylphenyl) pyrazin-2-yl)phenyl) acetamide of compound formula (I) with a solvent to get a clear solution;
ii) adding water and
iii) isolating the solid.

17. The process according to claim 16, wherein a solvent is selected from the group consisting of N-methyl-2-pyrrolidone, THF, acetone, methanol, dimethylacetamide, dimethylformamide, 1-4 dioxane and methyl ethyl ketone.

18. A process for preparation of the compound (S)-2-(4-(1,1-difluoro-2-hydroxypropyl)phenyl)-N-(4-(3-(2-ethylphenyl) pyrazin-2-yl)phenyl) acetamide (compound of formula (I)) or its pharmaceutically acceptable salt thereof, the process comprising the step of:
(a) reacting compound of formula (II) 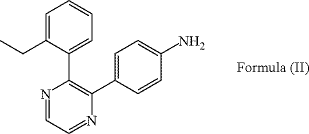 [SMILES: CCc1ccccc1-c1nccnc1-c1ccc(N)cc1]
with compound of formula (III) 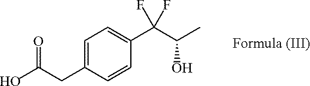 [SMILES: C[C@H](O)C(F)(F)c1ccc(CC(=O)O)cc1]
(b) optionally converting compound of formula (I) to its pharmaceutically acceptable salt.

19. The process according to claim 18, wherein the reaction between compound of formula (II) and compound of formula (III) is carried out in the presence of tetramethyluronium tetrafluoroborate (TBTU) or 1-[bis(dimethylamino)methylene]-1H-1,2,3-triazolo[4,5-b]pyridinium 3-oxid hexafluorophosphate) (HATU).

20. A process for preparation of the crystalline form of (S)-2-(4-(1,1-difluoro-2-hydroxypropyl)phenyl)-N-(4-(3-(2-ethylphenyl)pyrazin-2-yl)phenyl) acetamide, having a characteristic X- ray diffraction pattern comprising of the peaks expressed in terms of 2θ ±0.2: 11.22, 12.01, 15.88, 18.06, 19.28 and 19.97 comprising,
(a) reacting compound of formula (II) 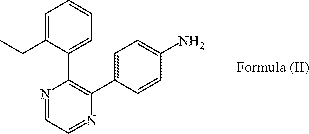 [SMILES: CCc1ccccc1-c1nccnc1-c1ccc(N)cc1]
with compound of formula (III) 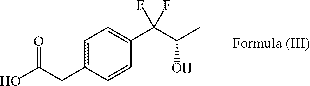 [SMILES: C[C@H](O)C(F)(F)c1ccc(CC(=O)O)cc1]
(b) treating (S)-2-(4-(1,1-difluoro-2-hydroxypropyl)phenyl)-N-(4-(3-(2-ethylphenyl) pyrazin-2-yl)phenyl) acetamide obtained in step (a) with a solvent;
(c) heating the reaction mass to get clear solution and gradually cooling and
(d) isolating the solid.

21. The process according to claim 20 wherein in step (b) a solvent is selected from acetonitrile, ethyl acetate, methanol, ethanol, isopropanol acetonitrile, acetone, methyl acetate, acetic acid, ethylene glycol and 1,4-dioxane.

22. A process for preparation of the amorphous form of (S)-2-(4-(1,1-difluoro-2-hydroxypropyl)phenyl)-N-(4-(3-(2-ethylphenyl) pyrazin-2-yl)phenyl) acetamide of compound formula (I) comprising,
(a) heating (S)-2-(4-(1,1-difluoro-2-hydroxypropyl)phenyl)-N-(4-(3-(2-ethylphenyl) pyrazin-2-yl)phenyl) acetamide of compound formula (I) to melt under vacuum; and
(b) cooling the step (a) product.
